SEQUENCE LISTING (19 sequences)

SEQ ID NO: 1 - DNA, 3996 bp, Homo sapiens
  misc_feature (1)..(3996)
  misc_feature (1)..(3996): TM9SF4/TUCAP 1 encoding sequence
agtttctgccaggagctaatatggcttccttagttacaccgttctctctcttcacctaatcagcgaccttactttcccagaccagactgtcgagcaggagctaagactccttttcccctctgctgaccgccactacaggagcggttgaagccagacgaccaccttgtggagttaaactccgtaaccagggagcaccacttccgctgacgtcattacggcgacacgtggatccaagatggcgacggcgatggattggttgccgtggtctttactgcttttctccctgatgtgtgaaacaagcgccttctatgtgcctggggtcgcgcctatcaacttccaccagaacgatcccgtagaaatcaaggctgtgaagctcaccagctctcgaacccagctaccttatgaatactattcactgcccttctgccagcccagcaagataacctacaaggcagagaatctgggagaggtgctgagaggggaccggattgtcaacacccctttccaggttctcatgaacagcgagaagaagtgtgaagttctgtgcagccagtccaacaagccagtgaccctgacagtggagcagagccgactcgtggccgagcggatcacagaagactactacgtccacctcattgctgacaacctgcctgtggccacccggctggagctctactccaaccgagacagcgatgacaagaagaaggaaaaagatgtgcagtttgaacacggctaccggctcggcttcacagatgtcaacaagatctacctgcacaaccacctctcattcatcctttactatcatcgggaggacatggaagaggaccaggagcacacgtaccgtgtcgtccgcttcgaggtgattccccagagcatcaggctggaggacctcaaagcagatgagaagagttcgtgcactctgcctgagggtaccaactcctcgccccaagaaattgaccccaccaaggagaatcagctgtacttcacctactctgtccactgggaggaaagtgatatcaaatgggcctctcgctgggacacttacctgaccatgagtgacgtccagatccactggttttctatcattaactccgttgttgtggtcttcttcctgtcaggtatcctgagcatgattatcattcggaccctccggaaggacattgccaactacaacaaggaggatgacattgaagacaccatggaggagtctgggtggaagttggtgcacggcgacgtcttcaggcccccccagtaccccatgatcctcagctccctgctgggctcaggcattcagctgttctgtatgatcctcatcgtcatctttgtagccatgcttgggatgctgtcgccctccagccggggagctctcatgaccacagcctgcttcctcttcatgttcatgggggtgtttggcggattttctgctggccgtctgtaccgcactttaaaaggccatcggtggaagaaaggagccttctgtacggcaactctgtaccctggtgtggtttttggcatctgcttcgtattgaattgcttcatttggggaaagcactcatcaggagcggtgccctttcccaccatggtggctctgctgtgcatgtggttcgggatctccctgcccctcgtctacttgggctactacttcggcttccgaaagcagccatatgacaaccctgtgcgcaccaaccagattccccggcagatccccgagcagcggtggtacatgaaccgatttgtgggcatcctcatggctgggatcttgcccttcggcgccatgttcatcgagctcttcttcatcttcagtgctatctgggagaatcagttctattacctctttggcttcctgttccttgttttcatcatcctggtggtatcctgttcacaaatcagcatcgtcatggtgtacttccagctgtgtgcagaggattaccgctggtggtggagaaatttcctagtctccgggggctctgcattctacgtcctggtttatgccatcttttatttcgttaacaagctggacatcgtggagttcatcccctctctcctctactttggctacacggccctcatggtcttgtccttctggctgctaacgggtaccatcggcttctatgcagcctacatgtttgttcgcaagatctatgctgctgtgaagatagactgattggagtggaccacggccaagcttgctccgtcctcggacaggaagccaccctgcgtgggggactgcaggcacgcaaaataaaataactcctgctcgtttggaatgtaactcctggcacagtgttcctggatcctggggctgcgtggggggcgggagggcctgtagataatcttgcgtttttcgtcatcttattccagttctgtgggggatgagtttttttgtgggttgctttttcttcagtgctaagaaagttccctccaacaggaactctctgacctgtttattcaggtgtatttctggtttggatttttttttccttctttgttttaacaaatggatccaggatggataaatccaccgagataagggttttggtcactgtctccacctcagttcctcagggctgttggccaccctatgactaactggaagaggacacgccagagcttcagtgaggtttccgagcctctccctgcccatcctcaccactgaggccacgacaaagcacagctccagctcggacagcaccctcagtgccagccagcctctgccagacctctctttccctcttctccccagcctcctccagggctgcccaaggcagggtttccagccaggcctcggggtcatcttttcaccaggagcaaacccaagtcttagttgctacaagaaaatcccctggaagtactgggggccaggttccccagacagcaggaattgcccctgttcagagcagccggagtttgctggaccacaaggaagaagagaagagacttgcagtgaactgtttttgtgccaagaaaccctggacctggggccaagtatttcccaagccaagcatccacttgtctgtgtctgggaagggatggccaaggccgctagggtccttacccctcaggatcactccccagccctttcctcaggaggtaccgctctccaaggtgtgctagcagtgggccctgcccaacttcaggcagaacagggaggcccagagattacagatcccctcctgtaagtggccaggcattctctccctgccctctctggcctctggggtcatactcacttctttagccagccccatcccctccaccccacacctgagttcttgcctcctccttttggggacacccaaaacactgcttgtgagaaggaagatggaaggtaagttctgtcgttctttccccaatccccaggaatggacaagaagccaacttagaaagaagggtctcacgtggctggcctggctcctccgtagacccctgttcttttcaacctctgcccacccgtgcatgtcatcacaaacatttgctcttaagttacaagagaccacatccacccagggattagggttcaagtagcagctgctaacccttgcaccagcccttgtgggactcccaacacaagacaaagctcaggatgctggtgatgctaggaagatgtccctcccctcactgccccacattctcccagtggctctaccagcctcacccatcaaaccagtgaatttctcaatcttgcctcacagtgactgcagcgccaagcggcatccaccaagcatcaagttggagaaaagggaacccaagcagtagagagcgatattggagtcttttgttcattcaaatcttggatttttttttttccctaagagattctctttttagggggaatgggaaacggacacctcataaagggttcaaagatcatcaatttttctgactttttaaatcattatcattattatttttaattaaaaaaatgcctgtatgcctttttttggtcggattgtaaataaatataccattgtcctactgaaaaaaaaaaaaaaaaaa

SEQ ID NO: 2 - protein, 642 aa, Homo sapiens
  MISC_FEATURE (1)..(642): TM9SF4/TUCAP-1 protein
MATAMDWLPWSLLLFSLMCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIHWFSIINSVVVVFFLSGILSMIIIRTLRKDIANYNKEDDIEDTMEESGWKLVHGDVFRPPQYPMILSSLLGSGIQLFCMILIVIFVAMLGMLSPSSRGALMTTACFLFMFMGVFGGFSAGRLYRTLKGHRWKKGAFCTATLYPGVVFGICFVLNCFIWGKHSSGAVPFPTMVALLCMWFGISLPLVYLGYYFGFRKQPYDNPVRTNQIPRQIPEQRWYMNRFVGILMAGILPFGAMFIELFFIFSAIWENQFYYLFGFLFLVFIILVVSCSQISIVMVYFQLCAEDYRWWWRNFLVSGGSAFYVLVYAIFYFVNKLDIVEFIPSLLYFGYTALMVLSFWLLTGTIGFYAAYMFVRKIYAAVKID

SEQ ID NO: 3 - DNA, 2391 bp, Homo sapiens
  misc_feature (1)..(2391): TM9SF2 encoding nucleic acid
cgcaaccggaactagccttctgggggccggcttggtttatctctggcggccttgtagtcgtctccgagactccccacccctccttccctcttgaccccctaggtttgattgccctttccccgaaacaactatcatgagcgcgaggctgccggtgttgtctccacctcggtggccgcggctgttgctgctgtcgctgctcctgctgggggcggttcctggcccgcgccggagcggcgctttctacctgcccggcctggcgcccgtcaacttctgcgacgaagaaaaaaagagcgacgagtgcaaggccgaaatagaactatttgtgaacagacttgattcagtggaatcagttcttccttatgaatacacagcgtttgatttttgccaagcatcagaaggaaagcgcccatctgaaaatcttggtcaggtactattcggggaaagaattgaaccttcaccatataagtttacgtttaataagaaggagacctgtaagcttgtttgtacaaaaacataccatacagagaaagctgaagacaaacaaaagttagaattcttgaaaaaaagcatgttattgaattatcaacatcactggattgtggataatatgcctgtaacgtggtgttacgatgttgaagatggtcagaggttctgtaatcctggatttcctattggctgttacattacagataaaggccatgcaaaagatgcctgtgttattagttcagatttccatgaaagagatacattttacatcttcaaccatgttgacatcaaaatatactatcatgttgttgaaactgggtccatgggagcaagattagtggctgctaaacttgaaccgaaaagcttcaaacatacccatatagataaaccagactgctcagggccccccatggacataagtaacaaggcttctggggagataaaaattgcctatacttactctgttagcttcgaggaagatgataagatcagatgggcgtctagatgggactatattctggagtctatgcctcatacccacattcagtggtttagcattatgaattccctggtcattgttctcttcttatctggaatggtagctatgattatgttacggacactgcacaaagatattgctagatataatcagatggactctacggaagatgcccaggaagaatttggctggaaacttgttcatggtgatatattccgtcctccaagaaaagggatgctgctatcagtctttctaggatccgggacacagattttaattatgacctttgtgactctatttttcgcttgcctgggatttttgtcacctgccaaccgaggagcgctgatgacgtgtgctgtggtcctgtgggtgctgctgggcacccctgcaggctatgttgctgccagattctataagtcctttggaggtgagaagtggaaaacaaatgttttattaacatcatttctttgtcctgggattgtatttgctgacttctttataatgaatctgatcctctggggagaaggatcttcagcagctattccttttgggacactggttgccatattggccctttggttctgcatatctgtgcctctgacgtttattggtgcatactttggttttaagaagaatgccattgaacacccagttcgaaccaatcagattccacgtcagattcctgaacagtcgttctacacgaagcccttgcctggtattatcatgggagggattttgccctttggctgcatctttatacaacttttcttcattctgaatagtatttggtcacaccagatgtattacatgtttggcttcctatttctggtgtttatcattttggttattacctgttctgaagcaactatacttctttgctatttccacctatgtgcagaggattatcattggcaatggcgttcattccttacgagtggctttactgcagtttatttcttaatctatgcagtacactacttcttttcaaaactgcagatcacgggaacagcaagcacaattctgtactttggttataccatgataatggttttgatcttctttctttttacaggaacaattggcttctttgcatgcttttggtttgttaccaaaatatacagtgtggtgaaggttgactgaagaagtccagtgtgtccagttaaaacagaaataaattaaactcttcatcaacaaagacctgtttttgtgactgccttgagttttatcagaattattggcctagtaatccttcagaaacaccgtaattctaaataaacctcttcccatacacctttcccccataagatctgtcttcaacactataaagcatttgtattgtgatttgattaagtatatatttggttgttctcaatgaagagcaaatttaaatattatgtgcatttgaa

SEQ ID NO: 4 - protein, 663 aa, Homo sapiens
  MISC_FEATURE (1)..(663): TM9SF2 protein
MSARLPVLSPPRWPRLLLLSLLLLGAVPGPRRSGAFYLPGLAPVNFCDEEKKSDECKAEIELFVNRLDSVESVLPYEYTAFDFCQASEGKRPSENLGQVLFGERIEPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTYSVSFEEDDKIRWASRWDYILESMPHTHIQWFSIMNSLVIVLFLSGMVAMIMLRTLHKDIARYNQMDSTEDAQEEFGWKLVHGDIFRPPRKGMLLSVFLGSGTQILIMTFVTLFFACLGFLSPANRGALMTCAVVLWVLLGTPAGYVAARFYKSFGGEKWKTNVLLTSFLCPGIVFADFFIMNLILWGEGSSAAIPFGTLVAILALWFCISVPLTFIGAYFGFKKNAIEHPVRTNQIPRQIPEQSFYTKPLPGIIMGGILPFGCIFIQLFFILNSIWSHQMYYMFGFLFLVFIILVITCSEATILLCYFHLCAEDYHWQWRSFLTSGFTAVYFLIYAVHYFFSKLQITGTASTILYFGYTMIMVLIFFLFTGTIGFFACFWFVTKIYSVVKVD

SEQ ID NO: 5 - DNA, 6140 bp, Homo sapiens
  misc_feature (1)..(6140): TM9SF3 encoding sequence
gaggaagaggctgaggaggcgcggggggcgggggaggctcaggagcgggcggtgacggcgacggcggcggcagaggaggcagcggctgggccgggccccgtgcgtctgtccgcgccccgtggatgcgaatcggccgcggcggaggcggcggcggcggaggaggcggcggcgggaggaggagtcggtgagccggctccgggccggaggggcgcggaggatgaggccgctgcctggcgctcttggcgtggcggcggccgccgcgctgtggctgctgctgctgctgctgccccggacccgggcggacgagcacgaacacacgtatcaagataaagaggaagttgtcttatggatgaatactgttgggccctaccataatcgtcaagaaacatataagtacttttcacttccattctgtgtggggtcaaaaaaaagtatcagtcattaccatgaaactctgggagaagcacttcaaggggttgaattggaatttagtggtctggatattaaatttaaagatgatgtgatgccagccacttactgtgaaattgatttagataaagaaaagagagatgcatttgtatatgccataaaaaatcattactggtaccagatgtacatagatgatttaccaatatggggtattgttggtgaggctgatgaaaatggagaagattactatctttggacctataaaaaacttgaaataggttttaatggaaatcgaattgttgatgttaatctaactagtgaaggaaaggtgaaactggttccaaatactaaaatccagatgtcatattcagtaaaatggaaaaagtcagatgtgaaatttgaagatcgatttgacaaatatcttgatccgtccttttttcaacatcggattcattggttttcaattttcaactccttcatgatggtgatcttcttggtgggcttagtttcaatgattttaatgagaacattaagaaaagattatgctcggtacagtaaagaggaagaaatggatgatatggatagagacctaggagatgaatatggatggaaacaggtgcatggagatgtatttagaccatcaagtcacccactgatattttcctctctgattggttctggatgtcagatatttgctgtgtctctcatcgttattattgttgcaatgatagaagatttatatactgagaggggatcaatgctcagtacagccatatttgtctatgctgctacgtctccagtgaatggttattttggaggaagtctgtatgctagacaaggaggaaggagatggataaagcagatgtttattggggcattccttatcccagctatggtgtgtggcactgccttcttcatcaatttcatagccatttattaccatgcttcaagagccattccttttggaacaatggtggccgtttgttgcatctgtttttttgttattcttcctctaaatcttgttggtacaatacttggccgaaatctgtcaggtcagcccaactttccttgtcgtgtcaatgctgtgcctcgtcctataccggagaaaaaatggttcatggagcctgcggttattgtttgcctgggtggaattttaccttttggttcaatctttattgaaatgtatttcatcttcacgtctttctgggcatataagatctattatgtctatggcttcatgatgctggtgctggttatcctgtgcattgtgactgtctgtgtgactattgtgtgcacatattttctactaaatgcagaagattaccggtggcaatggacaagttttctctctgctgcatcaactgcaatctatgtttacatgtattccttttactactattttttcaaaacaaagatgtatggcttatttcaaacatcattttactttggatatatggcggtatttagcacagccttggggataatgtgtggagcgattggttacatgggaacaagtgcctttgtccgaaaaatctatactaatgtgaaaattgactagagacccaagaaaacctggaactttggatcaatttctttttcataggggtggaacttgcacagcaaaaacaaacaaacgcaagaagagatttgggctttaacacactgggtactttgtgggtctctctttcgtcggtggcttaaagtaacatctatttccattgatcctaggttcttcctgactgctttctccaactgttcacagcaaatgcttggattttatgcagtaggcattactacagtacatggctaatcttcccaaaaactagctcattaaagatgaaatagaccagctctcttcagtgaagaggacaaatagtttatttaaagcatttgttccaataaaataaatagagggaaacttggatgctaaaattacatgaataggaatcttcctggcacttagtgtttctatgttattgaaaaatgatgttccagaaagattacttttttcctcttatttttactgccattgtcgacctattgtgggacatttttatatattgaatctgggttcttttttgactttttttttttcccaatccaacagcatccttttttttaaaagagagaattagaaaatattaaatcctgcatgtaatatatctgctgtcatcttagttggaccaacttcccatttatttatcttaaaactatacagttacatcttaattccatccaaagaagatacagtttgaagacagaagtgtactctctacaatgcaatttactgtacagttagaaagcaaagtgttaaatggagaagatacttgtttttattaaacattttgagatttagataaactacattttaactgaatgtctaaagtgattatcttttttccccccaagttagtcttaaatcttttgggtttgaatgaaggttttacataagaaattattaaaaacaaggggggtgggtaataaatgtatataacattaaataatgtaacgtaggtgtagattcccaaatgcatttggatgtacagatcgactacagagtacttttttcttatgatgattggtgtagaaatgtgtgatttgggtgggcttttacatcttgcctaccattgcatgaaacattggggtttcttcaaaatgtgtgtgtcatacttcttttgggaggggggttgttttcttctgtttattttctgagactcctacaggagccaaatttgtaatttagagacacttaattttgttaatcctgtctgggacacttaagtaacatctaaagcattattgctttagaatgttcaaataaaatttcctgaccaaattgttttgtggaaatagatgtgtttgcaatttgaagatatctttctgtccagaaggcaaaattaccgaatgccatttttaaaagtatgctataaactatgctactctcatacaggggacccgtattttaaaatctccagacttgcttacatctagattatccagcacaatcataaagtgaatgacaaaccctttgaatgaaattgtggcacaaaatctgttcaggttggtgtaccgtgtaaagtggggatggggtaaaagtggttaacgtactgttggatcaacaaataaaggttacagttttgtaagagaagtgatttgaatacatttttctggaactattcataatatgaagttttcctagaaccactgagtttctagtttaatagtttgctatgcaaatgaccacctaaaacaatactttatattgttatttttagaaagactcaaaacacctgtatttaaaccttaatatgaaaatcatgcaattaatagttacacaagatgttttcattacaaaatatgtacctatctattgatggactctacatcctatattgtgacatgtaagtcctttaaaaggtgaaaagtatgatttcttaccacttaagtatgattgatatgatccaacaaatttgatcagaagctgtaggtaaatcctcttctgaagccaaaatggtatattaaatataatttattggtacttccattttctcttccttcttacttgcctttaagatcttataaaaaagaaactaaaagttaatatttagttgcctatattatgtaaccttttaactatatataaagtacttttttggtttctttctcaccacttttattcaaaagtacttttaacataccaatacatagtctgtctgatgggagtataaattggacagtaaggttttgtcttaataaaatgaaatttgtttctcatgatatgaatcttgcaggtaagatgtagggtttattgaaaatgtgtgggttaaatgctttcaggtacaccaattctttctactaaattgagctctatttgaagttctttggaatctgtggtgaaaaataattttctgatttccaaatacattaagagcattaaatgaatattaatcacctttaaagtcttttagaaaaggacttgtattggtttttggctgcatagaggggttgaataagtgtatgtatgtgtgtgcgtgtgtgtgtgtcttcttaaagaagatgtaattcacaaatagtttagctccctagcgctcagttgtagaatagaaaatagaacattattcaagttaattgaaaggtgaggtttttatacccccactaatgctgtgtatctgtctttcgtttgttaacattatttgcttaatttctttcaactcacactttggataatactatcaaaaactaaggctaaacattccttgtgtatctttaagcatgcttctcctgaaatttaactacattagtagttgacatttgtatacatatatcctaatacaagagtaggataaggtggaaatgtaatggcctgagggatggtgaagcattcttttagtatttttcatcatgttgggctcctagattgtactggggttgcccataaatcaaaccccatactcttagaattcattatattatggtgatatccgaacctagtgaatggtatgcttgggtgttttccattgagagtggatggacctctttataaagttggttgctgcaaaatccagttcttccaaaagccactttatttagggtttattcacaagtcatatccattttggtacagtgtttgtttcctaatatttattaaccaccttataccaaatgtcttgcaaagaaatgttattaaaaccttgaatttttacaaatgtaaaaaacaaaaagtgtattaatgtatttgttcaggaaaagctacataccgaagggcttttgtatatgaattctgtggtggggagacccatttgtaatctatatggcagttccatctgggttttaagtttagatttcaccgtgtcttagtgcttcattctattggtttattggaacatgtaataaataggagtagtgatgtattaaaacacaagtattcattaatgttttatatcttcactaaaattctatagttatgaaactatcaatcaaggtgttatatttcagtcagaagtgaaaatttatgaagagtatttggaagtgtgtacagaaataaactagacttacaggtaggctagatcagaacgttaacatatgaacctgcagaaatctggtaagacttaaattcagtgtgaggaataactctagttctctcctatgagcatttcctaaaagccatctgatttggcattcttactggagctgcagacagaaatctacaaagacaaaagtaaacaaaattaagttattattccactgttaggaatggaaataaacttgtgaagtctgtttattttgaagtattggtgaactaggcttgctaattgataactgcagcagtttgtgtttactccagttcatcagcttaggtcatttgaaagatataagagcttaaggcaagaaagaaataacatggaattctatttgaaggacaacagaacattcttggaaaagcagctccagttggtttttcaactgtcaaacttgaatgtgtaagtccccacagagcatggacagtcggtgcagagttccaaggaaacaattattgcctgatgaccacttccattttgtatacactctttggttcgtataggccatattccaactggctttttagtaatagaaatccagtatataatgtatcaaatacaattgaggttctaacctagtgtgttaatttatctgaatttggatttttaaaaagtaataaaaagttaaatgta

SEQ ID NO: 6 - protein, 589 aa, Homo sapiens
  MISC_FEATURE (1)..(589): TM9SF3 protein
MRPLPGALGVAAAAALWLLLLLLPRTRADEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIHWFSIFNSFMMVIFLVGLVSMILMRTLRKDYARYSKEEEMDDMDRDLGDEYGWKQVHGDVFRPSSHPLIFSSLIGSGCQIFAVSLIVIIVAMIEDLYTERGSMLSTAIFVYAATSPVNGYFGGSLYARQGGRRWIKQMFIGAFLIPAMVCGTAFFINFIAIYYHASRAIPFGTMVAVCCICFFVILPLNLVGTILGRNLSGQPNFPCRVNAVPRPIPEKKWFMEPAVIVCLGGILPFGSIFIEMYFIFTSFWAYKIYYVYGFMMLVLVILCIVTVCVTIVCTYFLLNAEDYRWQWTSFLSAASTAIYVYMYSFYYYFFKTKMYGLFQTSFYFGYMAVFSTALGIMCGAIGYMGTSAFVRKIYTNVKID

SEQ ID NO: 7 - DNA, 2391 bp, Homo sapiens; identical to SEQ ID NO: 3
  misc_feature (1)..(2391): TM9SF1encoding sequence

SEQ ID NO: 8 - protein, 606 aa, Homo sapiens
  MISC_FEATURE (1)..(606): TM1SF1 protein
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFSAISVELYYIFATVWGREQYTLYGILFFVFAILLSVGACISIALTYFQLSGEDYRWWWRSVLSVGSTGLFIFLYSVFYYARRSNMSGAVQTVEFFGYSLLTGYVFFLMLGTISFFSSLKFIRYIYVNLKMD

SEQ ID NO: 9 - DNA, 20 bp, Artificial Sequence
  chemically synthesized
tgtgtgaaacaagcgccttc

SEQ ID NO: 10 - DNA, 19 bp, Artificial Sequence
  chemically synthesized
atgaggtggacgtagtagt

SEQ ID NO: 11 - DNA, 18 bp, Artificial sequence
  Chemically synthesized
ccatggagaaggctgggg

SEQ ID NO: 12 - DNA, 20 bp, Artificial sequence
  chemically synthesized
caaagttgtcatggatgacc

SEQ ID NO: 13 - protein, 262 aa, Homo sapiens
  MISC_FEATURE (1)..(262): Amino acid sequence corresponding to TM9SF4 aa 18-279
MCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIH

SEQ ID NO: 14 - protein, 16 aa, artificial sequence
  chemically synthesized
MGSDKIHHHHHHHHHH

SEQ ID NO: 15 - protein, 15 aa, Homo sapiens
  MISC_FEATURE (1)..(15): Amino acid sequence corresponding to TM9SF4 aa 221-235
ADEKSSCTLPEGTNS

SEQ ID NO: 16 - protein, 50 aa, Homo sapiens
  MISC_FEATURE (1)..(50): Amino acid sequence corresponding to TM9SF4 aa 303-352
RTLRKDIANYNKEDDIEDTMEESGWKLVHGDVFRPPQYPMILSSLLGSGI

SEQ ID NO: 17 - protein, 126 aa, Homo sapiens
  MISC_FEATURE (1)..(126): Amino acid sequence corresponding to TM9SF1 aa 90-215
ESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVER

SEQ ID NO: 18 - protein, 166 aa, Homo sapiens
  MISC_FEATURE (1)..(166): Amino acid sequence corresponding to TM9SF2 aa 106-271
EPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTY

SEQ ID NO: 19 - protein, 194 aa, Homo sapiens
  MISC_FEATURE (1)..(194): Amino acid sequence corresponding to TM9SF3 aa 29-222
DEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIH

(19) United States
(12) Patent Application Publication
(10) Pub. No.: US 2015/0044695 A1
(43) Pub. Date: Feb. 12, 2015

(54) Method and a Kit To Detect Malignant Tumors and Provide a Prognosis

(71) Applicants: Francesco Lozupone, Rome (IT); Mariantonia Logozzi, Rome (IT); Stefano Fais, Rome (IT); Antonio Chiesi, Vigasio (IT); Natasa Zarovni, Milan (IT)

(72) Inventors: Francesco Lozupone, Rome (IT); Mariantonia Logozzi, Rome (IT); Stefano Fais, Rome (IT); Antonio Chiesi, Vigasio (IT); Natasa Zarovni, Milan (IT)

(73) Assignee: HANSABIOMED OU, Tallin (EE)

(21) Appl. No.: 14/478,153

(22) Filed: Sep. 5, 2014

Related U.S. Application Data
(63) Continuation of application No. 13/290,207, filed on Nov. 7, 2011, now abandoned.
Application No. 13/290,207 is a continuation-in-part of application No. 12/321,412, filed on Jan. 21, 2009, now Pat. No. 8,617,806.
Application No. 12/321,412 is a continuation-in-part of application No. 12/321,821, filed on Jan. 26, 2009, now Pat. No. 8,097,407.
(60) Provisional application No. 61/062,528, filed on Jan. 25, 2008.
(60) Provisional application No. 61/062,453, filed on Jan. 25, 2008.

(30) Foreign Application Priority Data
Jan. 26, 2009 (AU) 2009207926
Jan. 26, 2009 (AU) 2009207927
Jan. 26, 2009 (BR) BR P 10906081-0
Jan. 26, 2009 (BR) BR P 10906683-7
Jan. 26, 2009 (CA) CA2713193
Jan. 26, 2009 (CA) CA2713196
Jan. 26, 2009 (EE) PCT/EE2009/000001
Jan. 26, 2009 (EE) PCT/EE2009/000002
Jan. 26, 2009 (JP) 2010543378
Jan. 26, 2009 (JP) 2010543379
Jan. 26, 2009 (MX) MX/A/2010/008164
Jan. 26, 2009 (MX) MX/A/2010/008174
Jan. 26, 2009 (RU) 2010135525
Jan. 26, 2009 (RU) 2010135526

(51) Int. Cl. G01N 33/574

(52) U.S. Cl. CPC G01N 33/57496; G01N 2333/4718; G01N 2333/4719
USPC 435/7.23

(57) ABSTRACT

A method and kit is provided to quantifying and qualifying exosomes in human cell derived samples or in body fluid based on expression of TM9-superfamily proteins on the exosomes. Furthermore, a method and a kit to diagnose malignant tumors is provided. The disclosure also provides a method to monitor tumor growth.

### PRIORITY CLAIM

This is a continuation application of U.S. patent application Ser. No. 13/290,207 filed on Nov. 7, 2011, claiming priority of U.S. patent application Ser. No. 12/321,412 filed on Jan. 26, 2009, U.S. provisional application No. 61/062,528 filed on Jan. 25, 2008, U.S. patent application Ser. No. 12/321,821 filed on Jan. 26, 2009, and U.S. provisional application No. 61/062,453 filed on Jan. 21, 2008, the contents of all of which are incorporated herein by reference in their entirety.

### SEQUENCE DATA

This application contains sequence data provided in computer readable form and as PDF-format. The PDF-version of the sequence data is identical to the computer readable format.

### COLOR DRAWINGS

This patent application contains at least one drawing executed in color. Copies of this patent or patent application publication with color drawing(s) will be provided by the Office upon request and payment of the necessary fee.

### FIELD OF THE INVENTION

The present invention relates generally to the field of cancer diagnosis and prognosis. More specifically, the invention relates to a method to diagnose malignant tumors by means of quantifying and qualifying exosomes in human body fluids.

### BACKGROUND

Exosomes are microvesicles of a size ranging between 30-120 nm, actively secreted through an exocytosis pathway normally used for receptor discharge and intercellular cross-talk. [1-2]

Several cell types including reticulocytes, dendritic cells, B cells, T cells, mast cells, epithelial cells, and embryonic cells are known to be capable of releasing exosomes, [3-4]; however, their increased amount in the peripheral circulation appears to be unique to pregnancy and to cancer. The primary source of circulating exosomes is the tumor. Tumor patients have been found to have very high levels of tumor derived exosomes in plasma, ascites and pleural effusions (5-8).

Molecular analyses of exosomes have demonstrated that all exosomes share certain common characteristics, including structure (delimited by lipid bilayer), size, density and general protein composition. Proteins commonly associated with all exosomes include cytoplasmic proteins such as tubulin, actin, actin-binding proteins, annexins and endolysosomal proteins such as LAMP1- and Rab-proteins, signal transduction proteins, MHC class I molecules, and heat-shock proteins (such as Hsp70 and Hsp90) [9-12], and tetraspanins (such as CD9, CD81 and lysosomal proteins CD63), some of which are commonly utilized as exosomal markers [11, 13]. The parent patent application Serial Number US2009/0220944 discloses for the first time that Rab5 is a universal exosomal marker. Indeed Rab5 is displayed on the exosomal membrane regardless of the origin of an exosome while not found on other membrane delimited vesicles present in human biofluids. While tumor-derived exosomes share some common exosomal proteins, they also exhibit an array of tumor related proteins, such as, but not limited to Caveolin-1, or tumor markers such as carcinoembryonic antigen or MART-1 [14-16]. The elevated presence of exosomes in blood and ascites fluids of cancer patients and the over-expression of certain biomarkers has lead investigators to propose a role for exosomes in tumor marker analysis. In U.S. provisional application No. 61/062,528 and in the subsequent non-provisional application Ser. No. 12/321,412, both of which are incorporated herein by reference, we proposed for the first time a method to quantify and qualify exosomes for use of diagnosis and prognosis of cancer. The method we suggested was based on ELISA based test using anti-Rab5, anti-CD63 and anti-caveolin 1 antibodies. Later U.S. Pat. No. 7,897,356 discloses a method of characterizing prostate cancer in a subject by identifying a biosignature on an exosome by determining presence or level of CD9, CD63, or CD81 protein from exosomes, determining presence or level of PSMA and/or PCSA protein from exosomes, determining the presence or level of B7H3 and/or EpCam protein from the exosomes and then comparing the levels with a reference.

Transmembrane 9 SuperFamily (TM9SF1, TM9SF2, TM9SF3, TM9SF4/TUCAP1) is a very closely related family of proteins with a high degree of homology, which have remained almost completely uncharacterized. This family of proteins is characterized by the presence of a large variable extracellular or lumenal N-terminal domain followed by nine putative transmembrane domains in its conserved C-terminal. The only data available describes TM9SF1 as a protein involved in the autophagic processes, and it seems to be differentially expressed in urinary bladder cancer [17-18]. There is no published data about TM9SF2. TM9SF3 has been reported to be upregulated in Paraclitaxel resistant breast cancer cells [19]. Finally TM9SF4 is involved in myeoloid malignancy [20]. U.S. Serial Number 2009/0191222 and corresponding provisional patent application No. 61/062,453, both of which are incorporated herein by reference, characterize this protein further and describe it as a new tumor associated protein, highly expressed in metastatic melanoma cells, while undetectable in normal skin cells and peripheral blood lymphocytes derived from healthy donors. Melanoma cells over-expressing TM9SF4-protein are characterized by a cannibal behavior. Tumor cell cannibalism is phenomenon characterized by the ability of tumor cannibal cells to phagocytose apoptotic cells, plastic beads, stained yeasts as well as live lymphocytes that has been observed in tumors of different histology, and is always related to a poor prognosis. On the basis of these data we called this protein TUmor Cannibalism Associated Protein (TUCAP1). Tucap1-gene (Tm9SF4) according to SEQ ID NO: 1 encodes the TUCAP1-protein that has an amino acid sequence according to SEQ ID NO: 2.

In US Serial Number 2009/0191222 and in the corresponding provisional patent application No. 61/062,453, both of which are incorporated herein by reference, it was shown that subcellular localization analysis suggests that this protein is mainly recovered in intracellular vesicles such as early endosomes since it co-localizes with early endosomal markers such as Rab5 and EEA1. Moreover the predicted structure of TM9SF4 as shown in US Serial Number 2009/0191222 and in the corresponding provisional patent application No. 61/062,453, makes it conceivable to hypothesize a role for this molecule as an ion channel or an ion channel regulatory protein involved in pH regulation of intracellular vesicles.

In US Serial Number 2009/0191222 and in the corresponding provisional patent application No. 61/062,453, both of which are fully incorporated herein by reference, it was proposed that TM9SF-proteins and especially TM9SF4, are new tumor markers. It was specifically suggested that TM9SF4 may also represent a potential new therapeutic target.

Given the increasing understanding of the role of exosomes in cancer progression and the fact that there is a persistent need to improve non-invasive cancer diagnostics and monitoring, methods and tools to detect and measure disease specific exosomes in human fluids represents an appealing strategy. Methods that are currently used to purify exosomes (ultracentrifugation, sucrose gradient) are either expensive or time consuming, requiring special devices or serial processing of fluids containing exosomes, while methods to detect and characterize exosomes are poorly quantitative (FACS and Western Blot). FACS (Fluorescence Activated Cell Sorter) is a suitable method to quantify cells, even of small size, while it is not suitable to quantify the amount of small vesicles such as exosomes (i.e. 50-100 nm). Moreover, the rough measurement of total mean fluorescence does not allow a precise quantification on how many microvesicles are actually present in the given sample. Furthermore, FACS-analysis does not allow simultaneous comparative analysis of different samples. In US Serial Number 2009/0220944 and in the corresponding provisional patent application No. 61/062,528, both of which are fully incorporated herein by reference, we disclosed a method to accurately quantify and characterize exosomes from human fluids. ExoTest™ is an ELISA-based method that couples immunocapturing to characterization and quantification of exosomes from fractionated or unfractionated human fluids of volume less than 2 ml.

Although some carcinoma cases can be classified reliably with current pathological criteria, there is still a significant subset of cases in which no consensus can be reached even among expert pathologists and reliable markers for both accurate diagnosis and prognosis are still lacking. Diagnostic ambiguity has significant adverse consequences for the patient. Misclassifying a tumor as benign may be fatal, and diagnosing a benign lesion as malignant may lead to unnecessary treatments. Currently there is no method to definitely resolve these ambiguities. Therefore, there is a clear need for a diagnostic test that could reduce these uncertainties.

### SUMMARY OF THE INVENTION

In this disclosure we show for the first time that Transmembrane 9 Super-Family proteins (TM9SF1, TM9SF2, TM9SF3 and TM9SF4/TUCAP-1) are being expressed on exosomes.

In this disclosure we also suggest the tumor exosomes associated proteins belonging to Transmembrane 9 Super-Family (i.e. TM9SF1, TM9SF2, TM9SF3 and TM9SF4) as new potential markers for the diagnosis and prognosis of cancer, based on ELISA based (ExoTest™) detection of these proteins on exosomes.

A central problem in obtaining useful in vivo data on exosomes is the low level of efficiency of currently available methods to obtain specific exosome preparations in order to quantify and characterize them from human body fluids, particularly from plasma. The body fluids may also be ascites, cerebral fluids, bone marrow, urine, faeces or bronco-alveolar washing. To provide a solution to these problems, this disclosure provides a simple a reliable method to detect and quantify exosomes from body fluids, especially from human plasma. According to this disclosure an ELISA based test (called ExoTest™) allows quantification and characterization of exosomes from human plasma of both healthy donors and tumor patients. The test described here allows characterization of exosomes purified from supernatants of human carcinoma including melanoma and colon carcinoma in vitro cultured cells and from plasma of healthy donors as compared to plasma of patients with different tumors. The test described here allows also quantification and characterization of exosomes from unfractionated samples of human fluids. The test provided here is an improvement of the test provided in US Serial Number 2009/0220944 and corresponding provisional application U.S. 61/062,528, both of which are incorporated herein by reference. The test disclosed here is designed to recognize exosomes carrying proteins or peptides belonging to TM9SF-superfamily. Monoclonal antibodies useful in the test described here are disclosed in the nonprovisional application entitles “Monoclonal antibodies, hybridomas, and methods for use” for Francesco Lozupone, Stefano Fais, Antonio Chiesi, Angela Pontillo, Paolo Sarmientos and Natasa Zarovni, which is filed on the same day as this application and which is fully incorporated herein by reference.

One object of this invention is to provide Transmembrane 9 Superfamily proteins (TM9SF) as novel exosome-associated markers.

Another object of this invention is to provide TM9SF proteins as specific markers of tumor derived exosomes from the plasma/serum of tumor patients.

Another object of this invention is to provide TM9SF4 (TUCAP 1)-protein as a specific marker of tumor derived exosomes from the plasma/serum of tumor patients.

Still another object of this invention is to provide a method and a tool for detection of TM9SF4 bearing tumor exosomes in the plasma/serum of human patients for diagnosis of human tumor malignancies and patients' follow up.

Yet another object of this invention is to provide TM9SF1, TM9SF2 and TM9SF3 as novel tumor markers based on their expression on tumor exosomes.

Yet another object of this invention is to provide a non-invasive test useful in clinical practice for diagnosis, follow up and screening of tumors, based on the utilization of proteins TM9SF1, TM9SF2, TM9SF3 and TM9SF4 related to the exosomes.

Still another object of this invention is to provide a technology for clinical research on tumors.

Another object of this invention is to provide tools to improve existing clinical tests based on proteins that are expressed on exosomes (e.g. TM9SF4 and the other TM9SF proteins as tumor markers).

Even further object of this invention is to provide specific antibodies for Transmembrane 9 Superfamily (TM9SF) proteins.

Yet another object of this invention is to provide an ELISA-based kit for detection of tumor related exosomes using antibodies against TM9SF proteins.

Another object of this invention is to provide an ELISA-based kit for detection of tumor related exosomes using antibodies against TM9SF4 (TUCAP1)-protein.

Another object of this invention is to provide a method to detect malignant melanoma tumors, gastro-intestinal tumors, prostate tumors, osteosarcoma tumors, B cell lymphoma tumors, breast tumors or ovary carcinoma tumors, lung tumors, liver tumors, and brain tumors.

It is an object of this invention to provide a method to quantify and qualify tumor-related exosomes in human cell derived samples or in body fluid, said method having the steps comprising: a) optionally purifying an exosome preparation from the human cell derived sample or body fluid; b) capturing exosomes of the purified exosome preparation or the human cell derived sample or body fluid with a primary antibody against a protein ubiquitously present on exosomes, said primary antibody being selected from the group consisting of: anti-tetraspanins, anti-annexins and anti-Rab-proteins; c) detecting tumor-related exosomes from the captured total exosomes with a detection antibody, said detection antibody being selected from the group consisting of antibodies against proteins belonging to the Transmembrane-9 Superfamily; d) allowing an enzyme linked secondary antibody to react with the detection antibody; e) adding substrate; and f) detecting the reaction.

Another object of this invention is to provide a method to diagnose a malignant tumor, said method comprising the steps of: a) taking a body fluid sample of a person suspected to have a tumor; b) optionally purifying an exosome preparation from the sample; c) capturing exosomes of the purified exosome preparation or the human cell derived sample or body fluid with a primary antibody against a housekeeping protein present on exosomes, said primary antibody being selected from the group consisting of: anti-tetraspanins, anti-annexins and anti-Rab-proteins; d) detecting tumor-related exosomes from the captured total exosomes with a detection antibody, said detection antibody being selected from the group consisting of antibodies against proteins belonging to Transmembrane-9 Superfamily; e) allowing an enzyme linked secondary antibody to react with the detection antibody; f) adding substrate; g) detecting the reaction; h) comparing reaction result with a reaction result obtained from an equally processed reference sample of a relevant body fluid from healthy donors, wherein a positive reaction and a level of positivity indicates a malignant tumor.

Still another object of this invention is to provide a test kit for quantifying and qualifying exosomes in human cell derived samples or in body fluid, said kit comprising: a) instructions to purify an exosome preparation from the human cell derived sample or from body fluid; b) a primary antibody preparation for capturing exosomes of the purified exosome preparation; c) a detection antibody preparation for detecting the bound exosomes, wherein detection antibody is selected from the group consisting of anti-TM9SF1, anti-TM9SF2, anti-TM9SF3 and anti-TM9SF4; d) an enzyme linked secondary antibody preparation for reaction with the detection antibody; e) a substrate for the enzyme; and f) a positive control consisting of a standard exosome preparation from human cancer cells that display a TM9SF protein of interest.

### BRIEF DESCRIPTION OF THE DRAWINGS

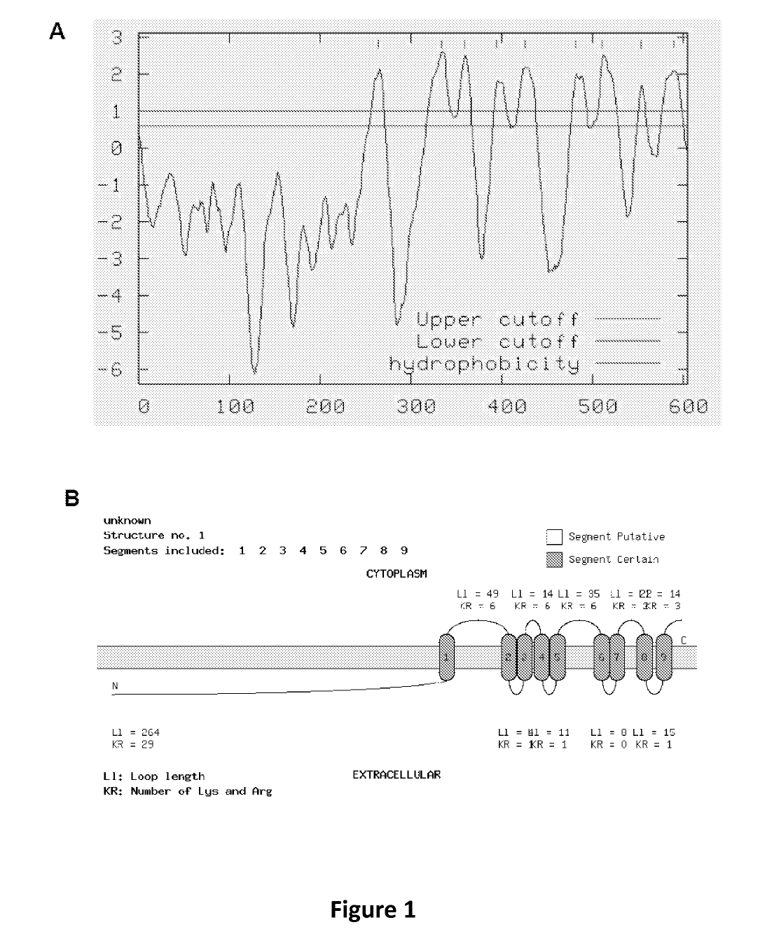

FIG. 1. Molecular structure of TM9SF4/TUCAP 1-protein:
A. Hydropathy profile of TUCAP-1 protein sequence. Hydrophobic regions are indicated above the line by positive values. Amino acid numbering is indicated on the abscissa. The hydrophilic stretch in the N-terminal region is followed by nine hydrophobic regions. The analysis was performed according to Claros and von Heijneb using TopPred prediction program.
B. Graphic representation of TUCAP-1 secondary structure according to TopPred predictor server is shown.

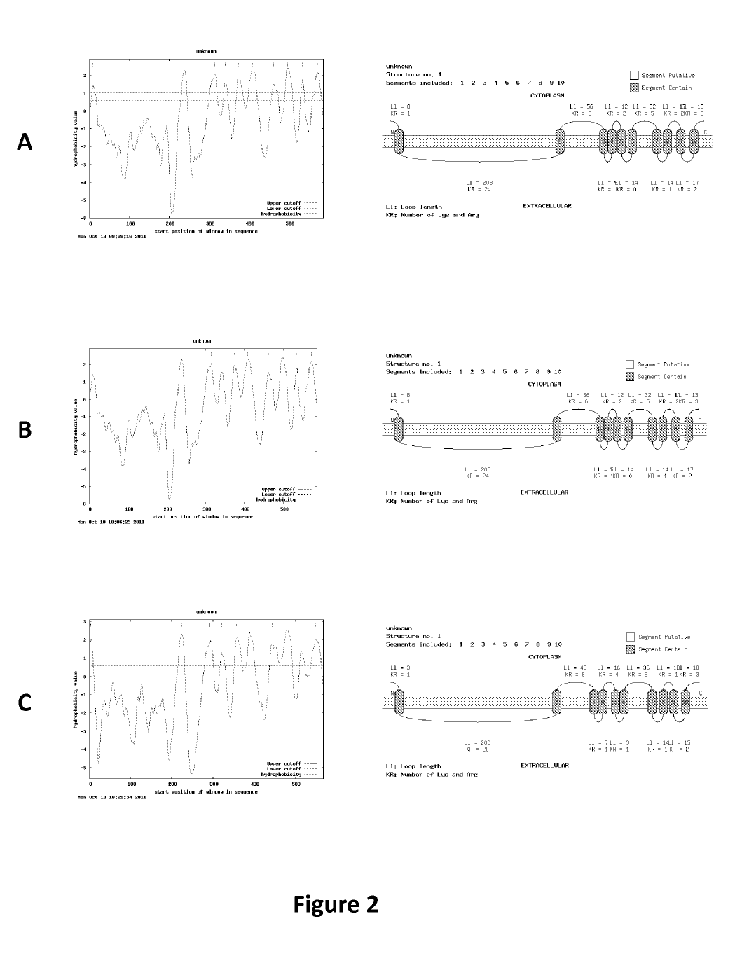

FIG. 2 Molecular structure of TM9SF1-3 proteins. The hydrophobic regions are indicated above the line by positive values. Amino acid numbering is indicated on the abscissa. The hydrophilic stretch in the N-terminal region is followed by nine hydrophobic regions. The analysis was performed according to Claros and von Heijneb using TopPred prediction server.
A. Hydropathy profile of TM9SF1 protein sequence (left) and Graphic representation of TM9SF1 secondary structure (right) according to TopPred predictor server.
B. Hydropathy profile of TM9SF2 protein sequence (left) and Graphic representation of TM9SF2 secondary structure (right) according to TopPred predictor server.
C. Hydropathy profile of TM9SF3 protein sequence (left) and Graphic representation of TM9SF3 secondary structure (right) according to TopPred predictor server.

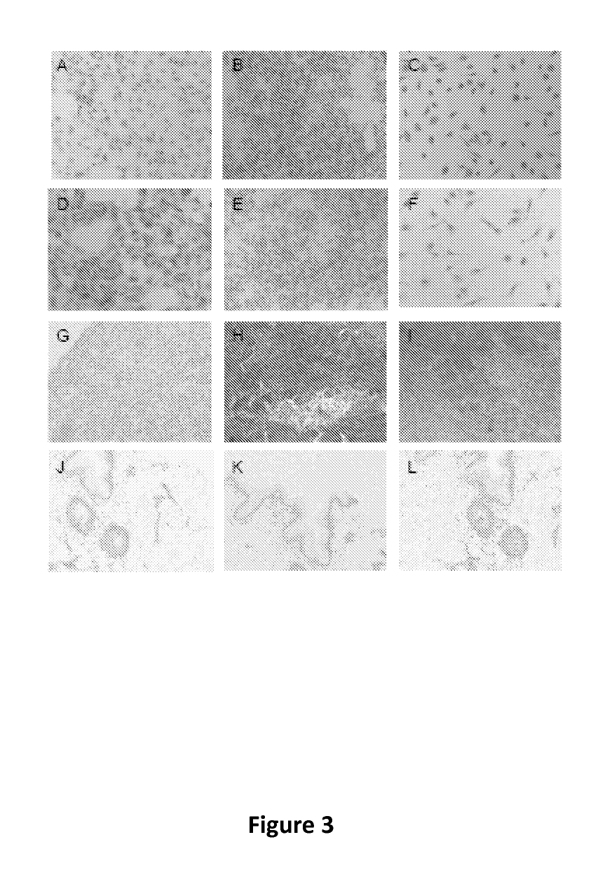

FIG. 3. Immuno-cytochemical and immuno-histochemical analysis of TUCAP-1:
A-C. Mice pre-immune serum immunocytochemical analysis of (A) MM2 cells; (B) peripheral blood lymphocytes; (C) in vitro differentiated macrophages.
D-F. TUCAP-1 immunocytochemical analysis of: (D) MM2 cells; (E) peripheral blood cells; (F) Macrophages.
G-I. Immunohistochemical analysis of malignant melanoma tissues stained with: (G) preimmune mouse serum; (H) TUCAP-1 immune serum; and (I) anti-GP100.
J-K. Immunohistochemical analysis of healthy skin stained with: (J) mouse preimmune serum, (K) TUCAP-1 immune serum, and (L) anti-ezrin antibody. Magnification 10×.

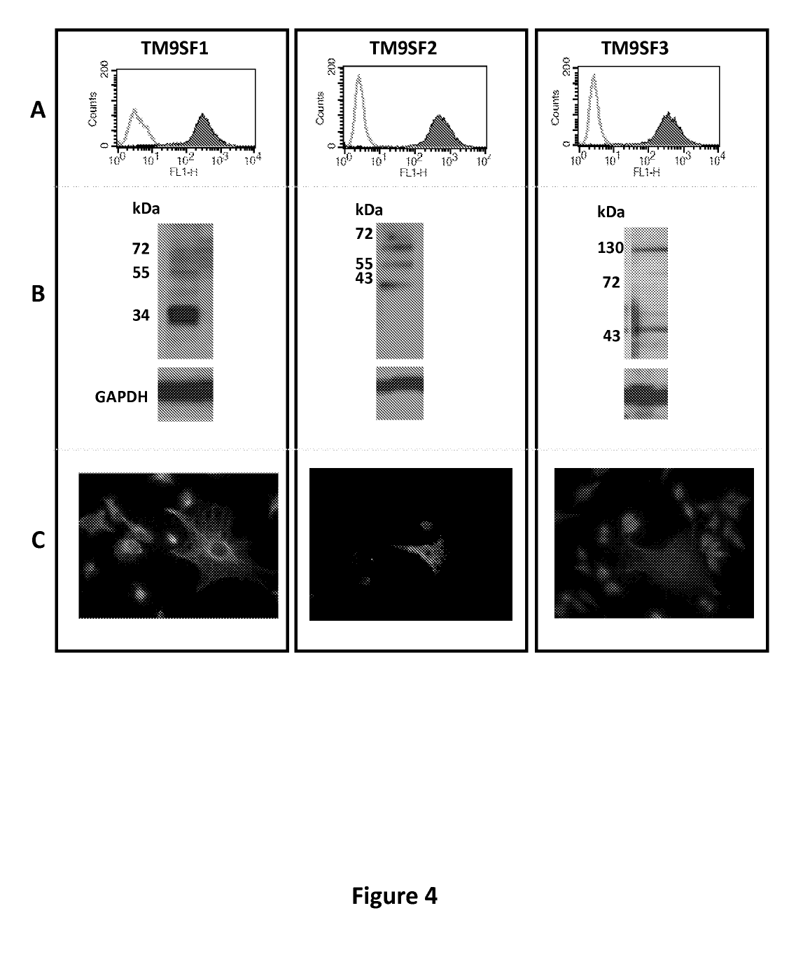

FIG. 4. Expression of TM9SF1, TM9SF2 and TM9SF3 on tumor cells. Expression analysis of anti-TM9SF1 (left) TM9SF2 (middle) and TM9SF3 (Right):
A. FACS-analysis of TM9SF proteins on MM1 cells, Green line: negative control, Purple: TM9SF proteins.
B. Western Blot analysis of Colo1 whole cell lysates immunoblotted with anti TM9SF proteins antibodies. GAPDH was used as housekeeping protein.
C. Immunofluorescence analysis of Colo cells stained TM9SF1 monoclonal antibodies. As negative controls control isotype antibodies were used. These results were obtained on both Colo colon carcinoma and MM1 melanoma and cell lines (not shown).

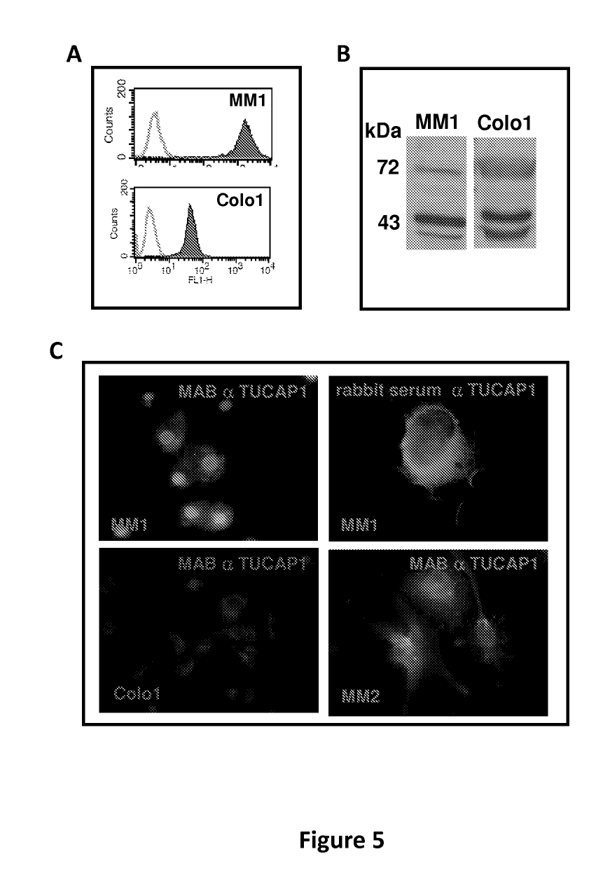

FIG. 5. Expression of TM9SF4 on MM1 and Cobol tumor cells:
A. FACS analysis of TM9SF4 expression on MM1 and Cobol cells, Green line: negative control, Purple: TM9SF4 protein.
B. Western Blot analysis of MM1 and Cobol whole cell lysates immunoblotted with rabbit polyclonal anti TM9SF4 serum.
C. Immunofluorescence analysis of MM1 cells stained with either monoclonal or polyclonal TM9SF4 antibodies and MM2 and Colo1 cells stained with monoclonal anti TM9SF4 antibody. As negative controls control isotype antibodies were used.

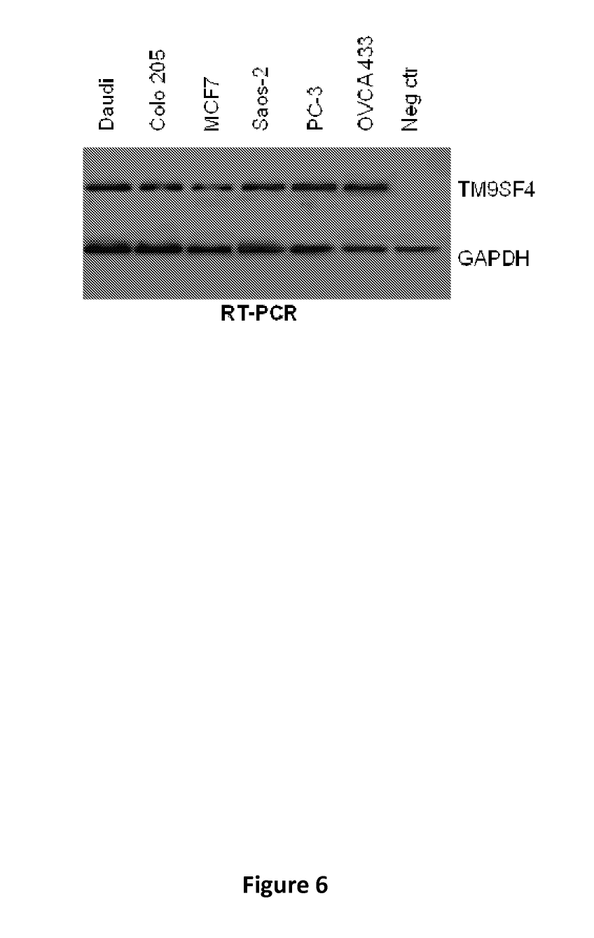

FIG. 6 RT-PCR analysis of TM9SF4 on different tumor cell lines:
The expression of TUCAP1/TM9SF4 was evaluated by RT-PCR on different cell lines. The data is representative of B lymphoma (Daudi); Colon Carcinoma (Colo 205); breast carcinoma (MCF7); Osteosarcoma (Saos-2); prostate cancer (PC-3); and ovary carcinoma (OVCA 433). Metastatic melanoma MM1 cells as positive control were used. As negative control template without reverse transcriptase was used. GAPDH was used as a housekeeping gene.

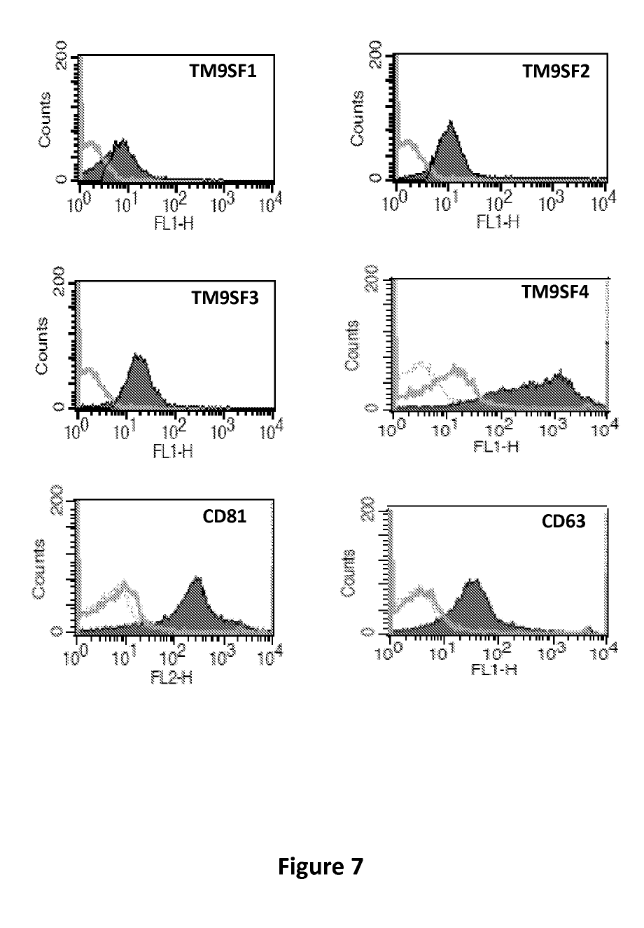

FIG. 7. FACS-analysis of the expression of the TM9SF proteins on exosomes. Exosomes purified from the supernatant of Colo1-cells and coated to latex beads were analyzed for TM9SF1, TM9SF2, TM9SF3 and TM9SF4 expression. As positive controls CD63 and CD81 expression was evaluated. As secondary antibody goat anti-mouse AlexaFluor488 conjugated secondary antibody was used. As negative control exosomes coated latex beads stained with irrelevant immunoglobulins and secondary antibody were used. Green line represents negative control and filled purple represents positive cells.

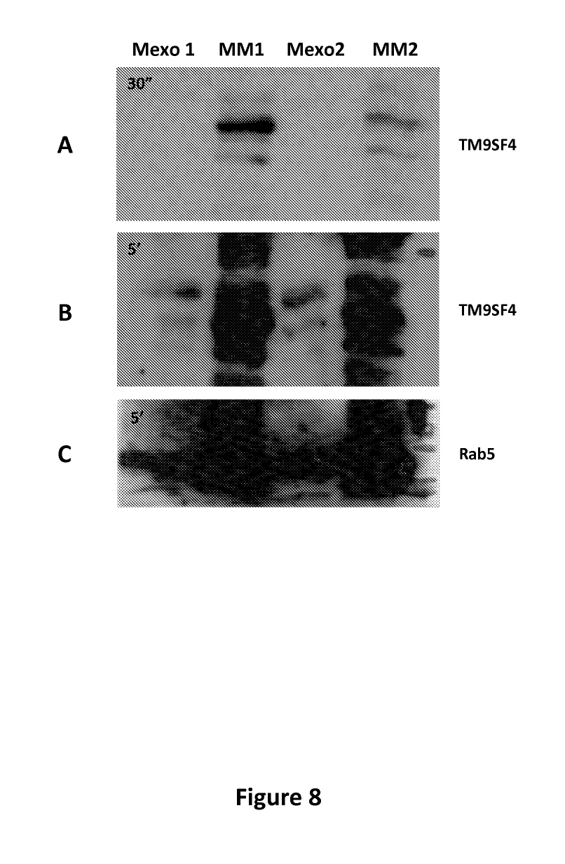

FIG. 8. Western Blot analysis of tumor exosomes deriving from two metastatic melanoma cell lines MM1 and MM2 immunoblotted with TM9SF4 and Rab5.
A. Whole lysates of exosomes deriving from MM1 and MM2 melanoma cells (respectively mexo1 and mexo2) and MM1 and MM2 cells total lysates immunoblotted for TM9SF4 detection.
B. A longer exposition of the same membrane showing that the protein is detectable on exosomal lysates.
C. Whole lysates of MM1 and MM2 cells deriving exosomes and MM1 and MM2 cells total lysates immunoblotted for Rab5 detection as a housekeeping protein.

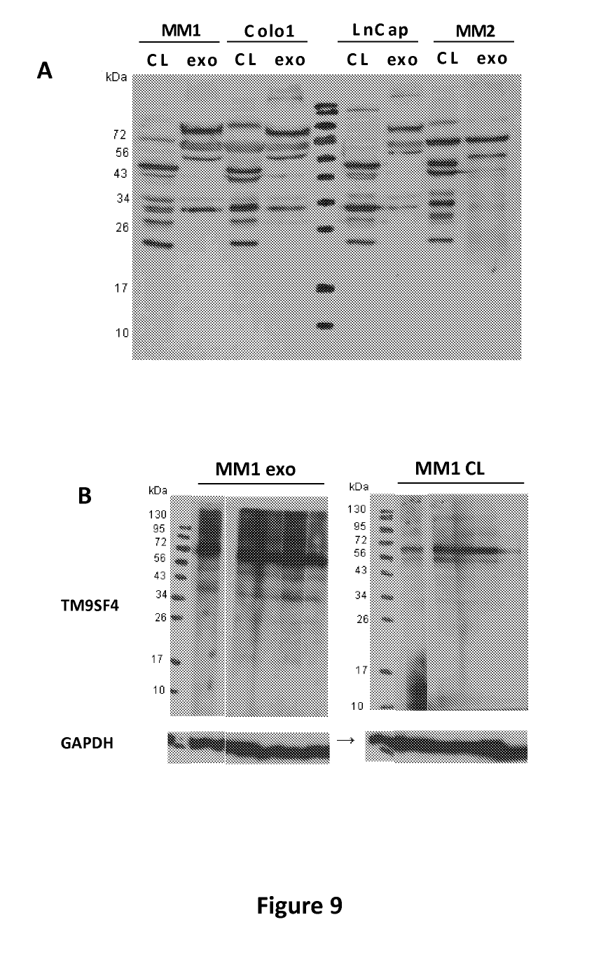

FIG. 9. Expression of TM9SF4 (TUCAP1) on tumor cells and correspondent exosomes detected with polyclonal and monoclonal antibodies anti-TUCAP1.
A. Western Blot analysis of Colo1 (colon carcinoma), MM1 and MM2 (metastatic melanoma) and LnCap (prostate cancer) whole cell lysates and exosome fractions immunoblotted with rabbit polyclonal anti TM9SF4 serum. Equal amounts of material (40 μg) were loaded per lane and GAPDH was used as housekeeping protein. Despite unspecific binding expected size bands (˜72 and 42 KDa) were observed in all cell lysates and exosomes.
B. Western Blot analysis of MM1 (metastatic melanoma) whole cell lysates and exosome fractions immunoblotted with monoclonal anti TM9SF4 antibodies. Equal amounts were analysed per lane (100 μg) and GAPDH was used as housekeeping protein. Enrichment of TUCAP, in particular some isoforms/band, has been observed in exosomal fractions.

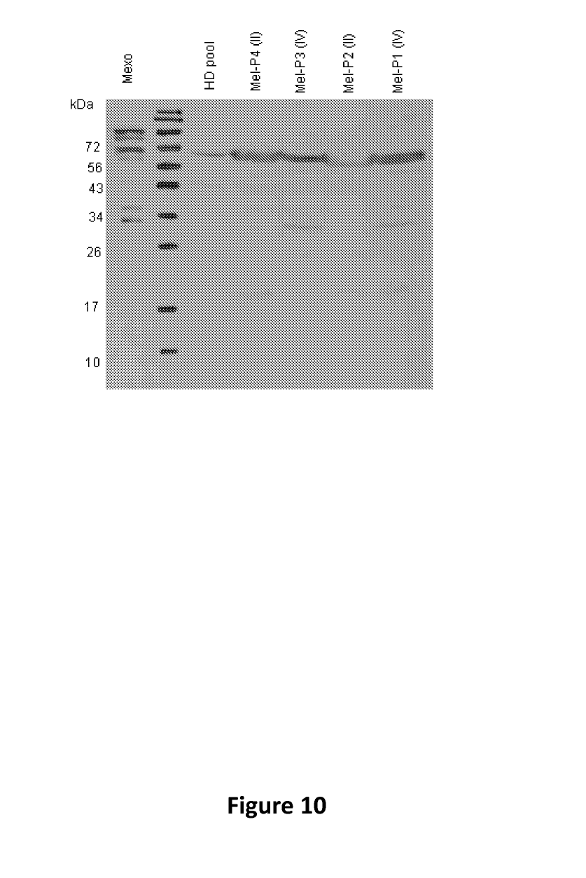

FIG. 10. Western Blot analysis of exosomes purified from plasma samples from patients with melanoma and from healthy donors pool (HD) immunoblotted with rabbit polyclonal anti TM9SF4 antibody. MM1 exosomes (mexo) were used as a control (40 μg) and exosomes purified from 0.5 ml of plasma were loaded per lane. Evident increase of TUCAP-1 on tumor patients exosomes was observed when compared to Healthy donor (HD) sample. Increased expression of exosome associated TUCAP along with a specific presence of distinct bands, is observed in advanced (stage III/IV) patients differently from early cancer patients (stage I/II).

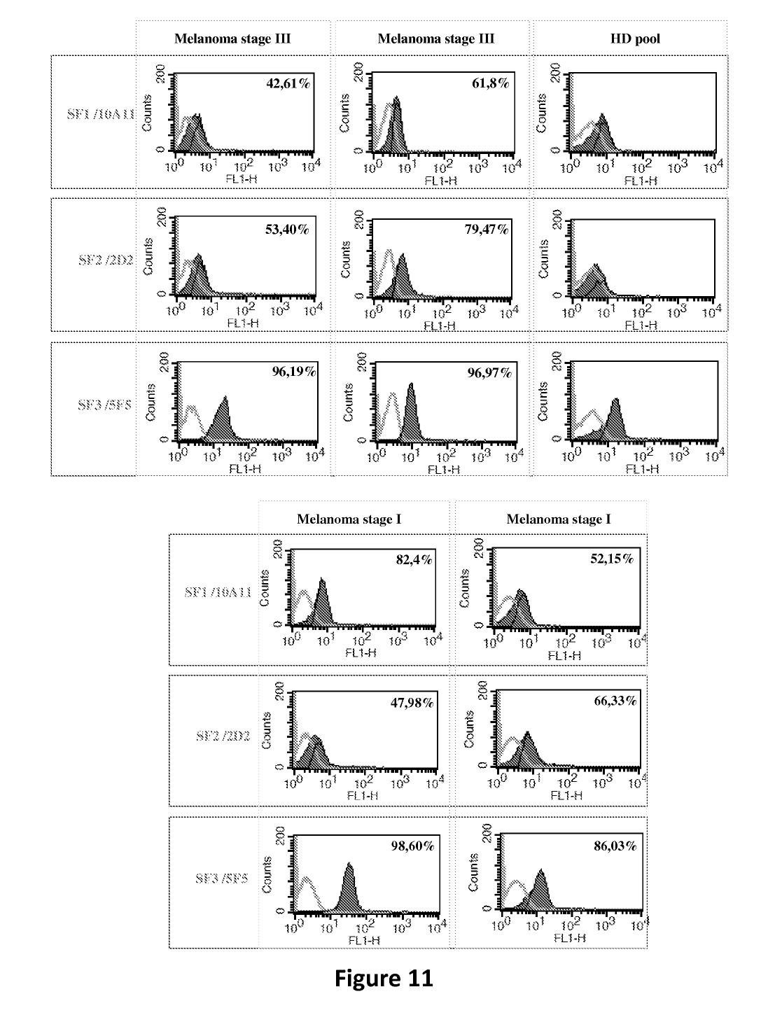

FIG. 11 FACS analysis of the expression of the TM9SF proteins on plasma exosomes from melanoma patients and healthy donors. Exosomes purified from plasma samples from four melanoma patents, two with an early decease and two with advanced tumors, were purified and coated to latex beads and analyzed for TM9SF1, TM9SF2, TM9SF3 expression. As positive control CD63 expression was detected (not shown). For every test exosomes purified from 0.25 ml of plasma were used. As secondary antibody goat anti-mouse AlexaFluor488 conjugated_secondary antibody was used. As negative control exosomes coated latex beads stained with irrelevant immunoglobulins and secondary antibody were used. Green line represents negative control and filled purple represents positive beads.

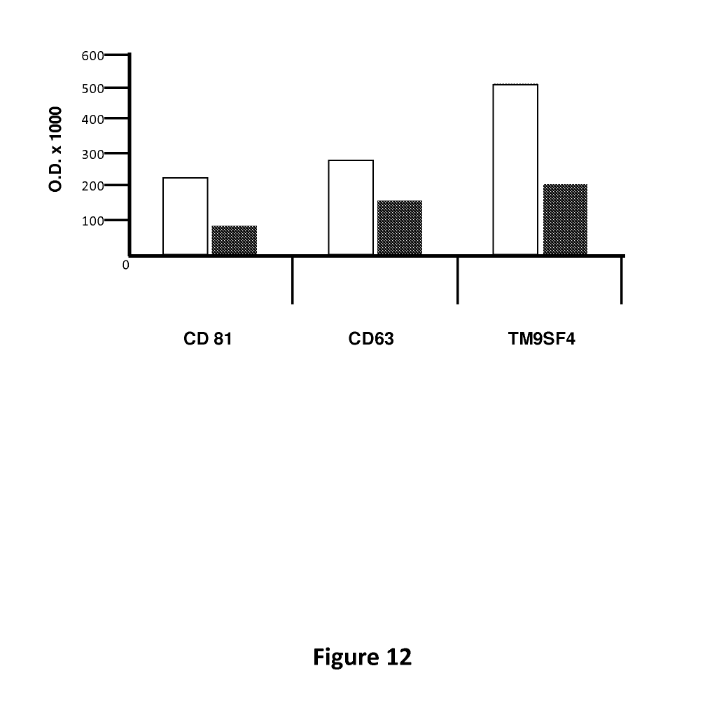

FIG. 12. ExoTest analysis of purified exosomes from cultured cells supernatants. Exosomes purified by ultracentrifugation of supernatants of MM1 and MM2 metastatic melanoma cell lines (white and black bars, respectively) analyzed by ExoTest for the detection of TM9SF4. As positive controls CD81 and CD63 as exosomes detection antigens were used. Exosomes levels are expressed as OD (wavelength 450 nm)×1000.

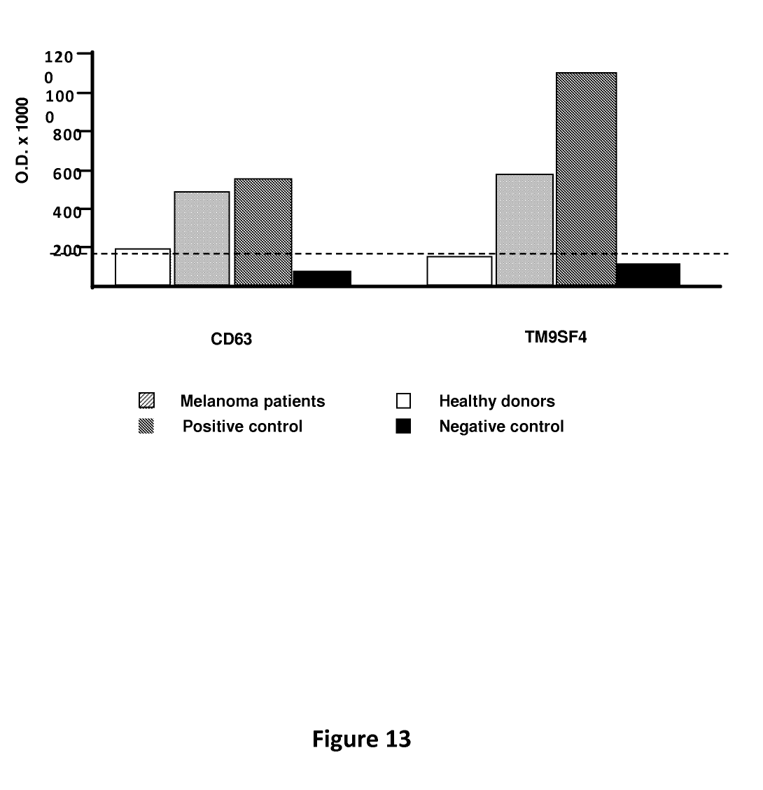

FIG. 13. ExoTest analysis for TM9SF4-expression on exosomes derived from plasma samples. Exosomes purified from five samples of healthy donors and five samples of melanoma patients were analyzed by ExoTest for the detection of TM9SF4, CD81 and CD83. As positive controls the same antigens were detected on 50 μg of exosomes purified from MM1 supernatants. Negative control: Rab5 coated wells plus detecting antibodies (antibodies to TM9SF4 or CD81 or CD63) and a secondary antibody. Exosomes levels are expressed as OD (wavelength 450 nm)×1000.

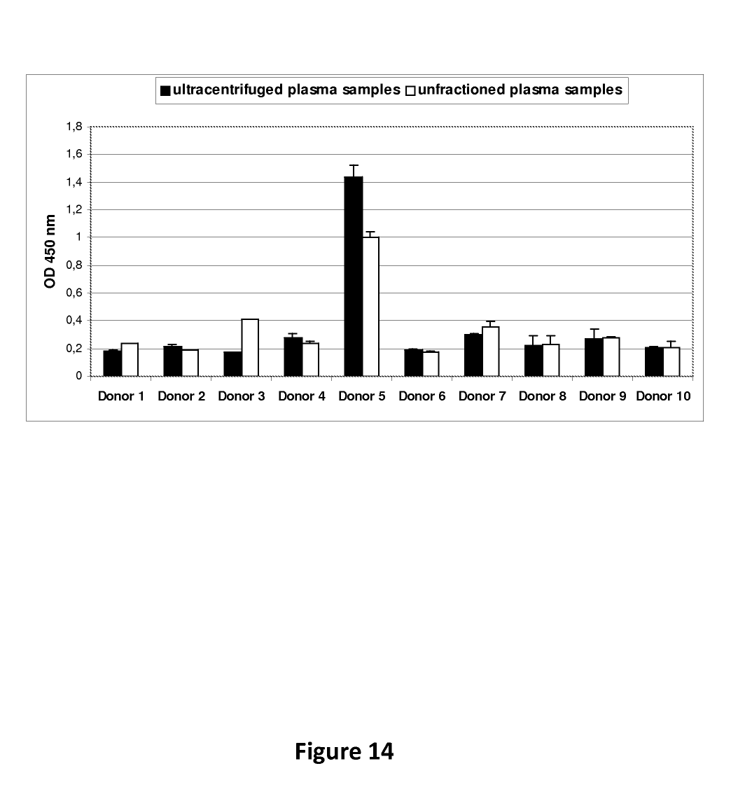

FIG. 14. Comparison of quantification of exosomes by CD63 Exotest detection from human plasma samples either upon purification via ultracentrifugation or from corresponding unfractionated samples. Set of ten healthy donors plasma samples were either purified by standard ultracentrifugation protocol or were precleared by microfiltration through 0.22 and 0.1 μm filters and concentrated in spin concentrators (Millipore), Material corresponding to 0.5 ml of original plasma sample was analyzed per well. Measured CD63 values are shown as OD (wavelength 450 nm) readings subtracted by negative control. As a negative control, sample buffer was used for purified exosomes while exosomes depleated plasma was used as control (blank) for unfractioned plasma samples.

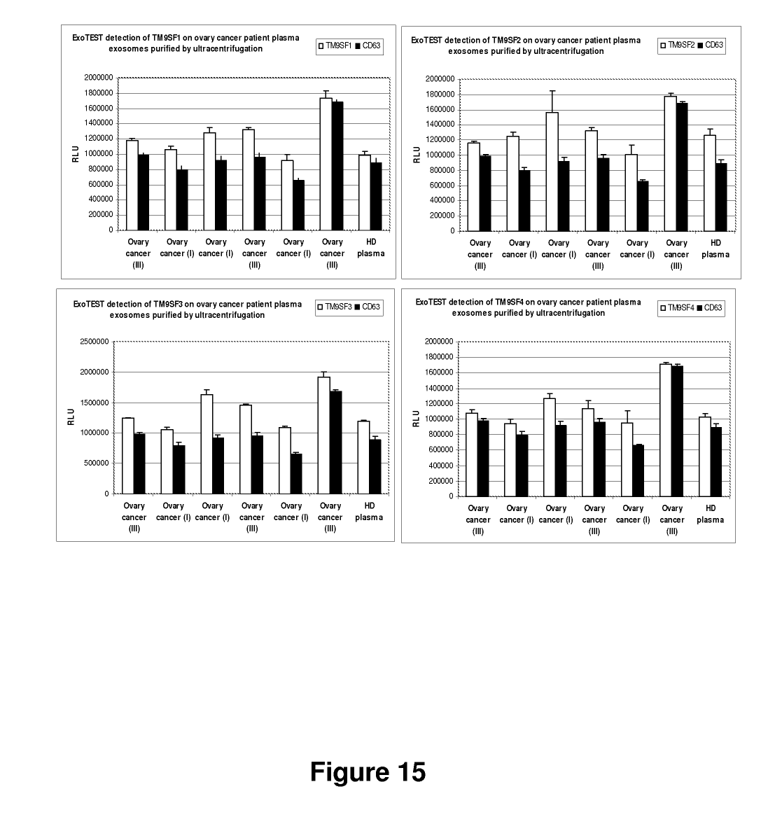

FIG. 15. ExoTEST detection and quantification of TUCAP-1(TM9SF4), TM9SF1, TM9SF2 and TM9SF3 on tumor patient plasma exosomes. Plasma exosomes were purified by ultracentrifugation from samples obtained from patients with ovary cancer. Every group comprised patients in advanced stage (III) and with an early disease (stage I) according to the information provided within patient sample collection sheet. No other follow-up information was available at the time experiment was performed. Purified exosomes were loaded onto ExoTEST plate, 0.5 ml of plasma derived exosomes per each well, and analyzed for the expression of CD63 for overall exosomes quantification in the sample, and for the expression of TM9SF1-4 proteins for the quantification of tumor related exosomes in the sample. ExoTEST was performed according to the standard protocol in white plates for chemiluminometric detection. The reaction is developed by addition of chemiluminiscence substrate and immediate reading of RLU (relative light units) values at the luminometer at 200 ms. Besides patients samples also plasma exosomes purified from a pool of healthy donors (HD plasma) were analyzed on the plate for the same exosomal markers while exosomes from HBM-Colo1 cells were used as positive control (not shown).

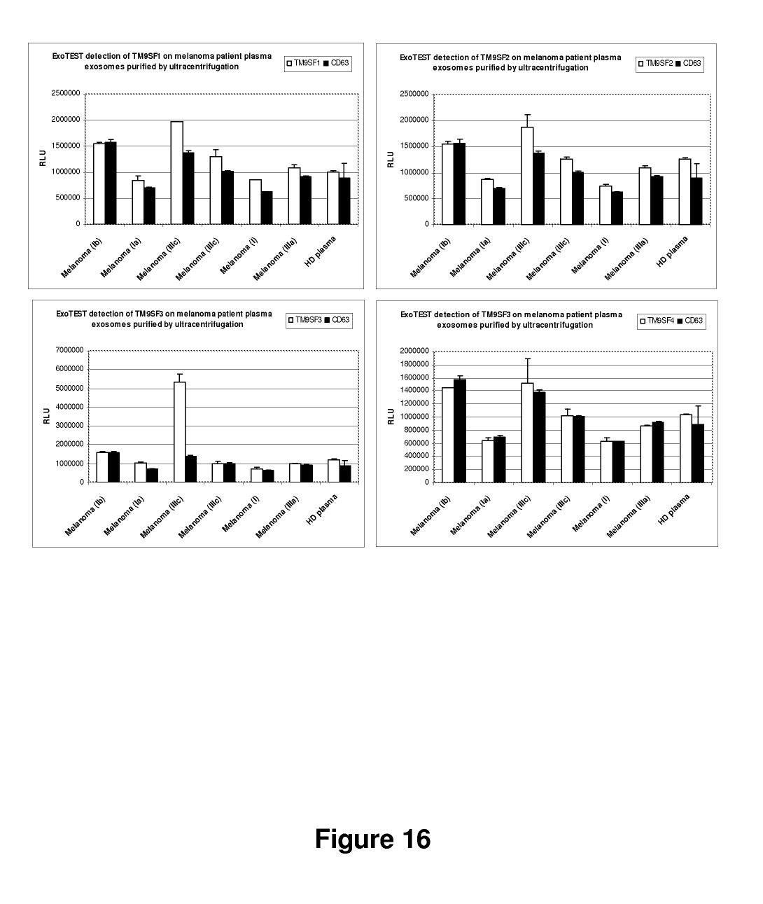

FIG. 16. ExoTEST detection and quantification of TUCAP-1(TM9SF4), TM9SF1, TM9SF2 and TM9SF3 on tumor patient plasma exosomes. Plasma exosomes were purified by ultracentrifugation from samples obtained from patients with melanoma. Every group comprised patients in advanced stage (III) and with an early disease (stage I) according to the information provided within patient sample collection sheet. No other follow-up information was available at the time experiment was performed. Purified exosomes were loaded onto ExoTEST plate, 0.5 ml of plasma derived exosomes per each well, and analyzed for the expression of CD63 for overall exosomes quantification in the sample, and for the expression of TM9SF1-4 proteins for the quantification of tumor related exosomes in the sample. ExoTEST was performed according to the standard protocol in white plates for chemoluminometric detection. The reaction is developed by addition of chemiluminiscence substrate and immediate reading of RLU (relative light units) values at the luminometer at 200 ms. Besides patients samples also plasma exosomes purified from a pool of healthy donors (HD plasma) were analyzed on the plate for the same exosomal markers while exosomes from HBM-Colo1 cells were used as positive control (not shown).

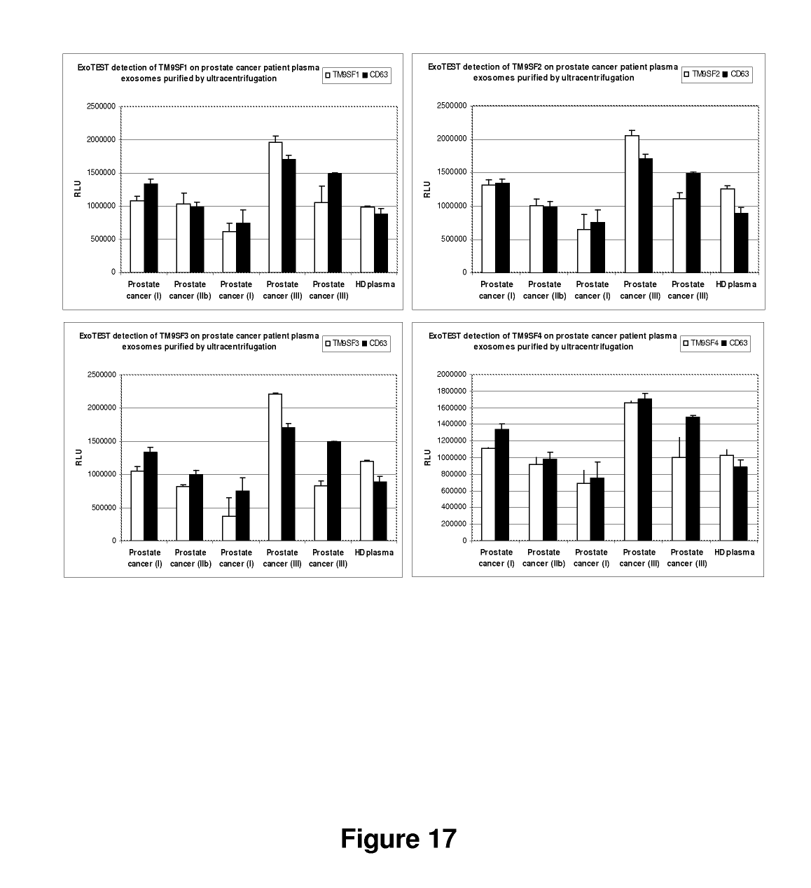

FIG. 17. ExoTEST detection and quantification of TUCAP-1(TM9SF4), TM9SF1, TM9SF2 and TM9SF3 on tumor patient plasma exosomes. Plasma exosomes were purified by ultracentrifugation from samples obtained from patients with prostate cancer. Every group comprised patients in advanced stage (III) and with an early disease (stage I) according to the information provided within patient sample collection sheet. No other follow-up information was available at the time experiment was performed. Purified exosomes were loaded onto ExoTEST plate, 0.5 ml of plasma derived exosomes per each well, and analyzed for the expression of CD63 for overall exosomes quantification in the sample, and for the expression of TM9SF1-4 proteins for the quantification of tumor related exosomes in the sample. ExoTEST was performed according to the standard protocol in white plates for chemoluminometric detection. The reaction is developed by addition of chemiluminiscence substrate and immediate reading of RLU (relative light units) values at the luminometer at 200 ms. Besides patients samples also plasma exosomes purified from a pool of healthy donors (HD plasma) were analyzed on the plate for the same exosomal markers while exosomes from HBM-Colo1 cells were used as positive control (not shown).

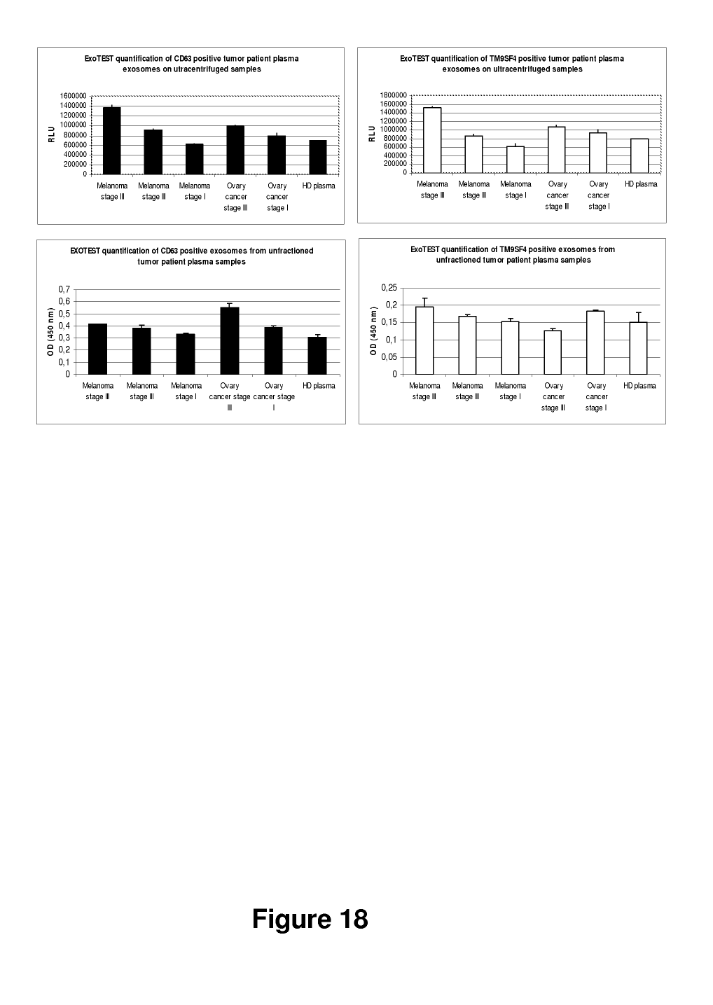

FIG. 18. Comparative quantification of exosome associated tumor markers by ExoTEST on purified plasma exosomes vs. unfractioned plasma samples from patients with melanoma, and ovary cancer. Same set of patients was analyzed for the CD63 expression for the purpose of overall exosomes quantification and for the presence and enrichment of TM9SF4 positive exosomes either upon purification via ultracentrifugation or from corresponding unfractioned samples. Standard ExoTEST and sample purification/preclearing protocols were used and the ExoTEST developed by colorimetric or luminometric detection and results shown as OD 450 nm or RLU readings.

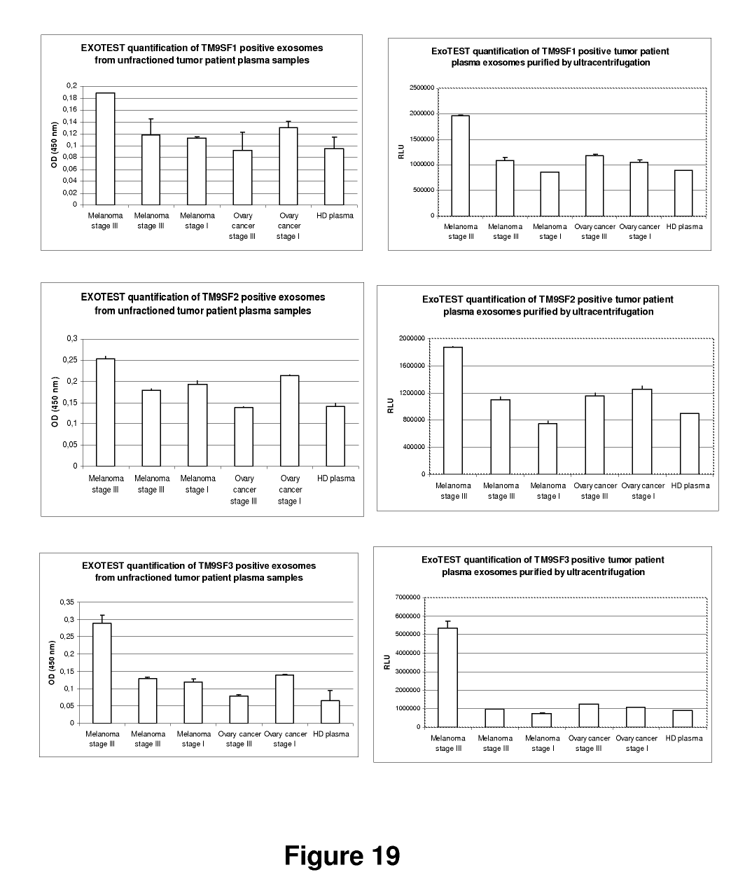

FIG. 19. Comparative quantification of exosome associated tumor markers by ExoTEST on purified plasma exosomes vs. unfractioned plasma samples from patients with melanoma, and ovary cancer. Same set of patients was analyzed for the CD63 expression for the purpose of overall exosomes quantification and for the presence and enrichment of TM9SF1-3 positive exosomes either upon purification via ultracentrifugation or from corresponding unfractioned samples. Standard ExoTEST and sample purification/preclearing protocols were used and the ExoTEST developed by colorimetric or luminometric detection and results shown as OD 450 nm or RLU readings

### DETAILED DESCRIPTION OF THE INVENTION

### Definitions

Antibodies—The term “antibodies” is used in this disclosure to include polyclonal and monoclonal antibodies. If monoclonal antibodies are specifically meant the term ‘monoclonal antibodies’ is used.

Housekeeping protein as used herein means a protein ubiquitously expressed on all exosomes in both physiological and pathological conditions.

TM9SF as user herein, the term “TM9SF” means the Transmembrane 9 Super Fmaily. The Transmembrane 9 Super Family is a very close related family of proteins with a high degree of homology. Proteins belonging to the Super Family include TM9SF1, TM9SF2, TM9SF3, and TM9SF4 (also called TUCAP1).

TM9SF1-protein as used herein refers to a protein encoded by tm9sf1-gene located in chromosome14 (map 14q11.2) and having nucleic acid sequence according to SEQ ID NO: 7. TM9SF1-protein has amino acid sequence according to SEQ ID NO:8.

TM9SF2-protein as used herein refers to a protein encoded by tm9sf2-gene located in chromosome 13 (map 13q32.3) and having nucleic acid sequence according to SEQ ID NO:3. TM0SF2-protein has amino acid sequence according to SEQ ID NO: 4.

TM9SF3-protein as used herein refers to a protein encoded by tm9s13-gene located in chromosome 10 (map 10q24.1) and having nucleic acid sequence according to SEQ ID NO: 5. TM9SF2-protein has amino acid sequence according to SEQ ID NO:6.

TM9SF4-protein, as used in this application is Human Genome Project-nomenclature and a synonym of TUCAP-1 protein. The protein is encoded by tucap 1-gene (tm9sf4-gene) located in chromosome 20q11.21 and having nucleic acid sequence according to SEQ ID NO: 1. TM9SF4-protein has an amino acid sequence according to SEQ ID NO:2. The structure of the protein is shown in FIG. 1.

TUCAP 1-protein (Tumor Associated Cannibal Protein), as used in this application is a synonym of TM9SF4 (Human Genome Project nomenclature). The protein is encoded by tucap 1-gene (tm9sf4-gene) located in chromosome 20q11.21 and having nucleic acid sequence according to SEQ ID NO:1 TUCAP-protein has an amino acid sequence according to SEQ ID NO:2 ExoTest™ is a trademarked ELISA-based test that was first described and claimed in the U.S. provisional patent application No. 61/062,528 and subsequent US Serial Number 2009/0220944, both of which are incorporated herein by reference. ExoTest platform comprises ELISA plates pre-coated with antibodies against housekeeping exosome proteins enabling specific capture of exosomes from different biological samples, including cell culture supernatants and human biological fluids. Quantification and characterization of exosomal proteins is subsequently performed by using appropriate detection antibodies against exosome associated antigens that can be either common for all exosomes or cell type- or cell condition specific. By employing different combinations of capture and detection antibodies ExoTest can be customized for assessing multiple antigens in a total exosome population as well as enrichment with cell/tissue specific exosomes from body fluids. The assay provides immediate readouts, namely origin, quantity and molecular composition of isolated exosomes. For the samples of interest, RNA (mRNA or miRNA) can be extracted and analysed from captured exosomes.

Exosomes are microvesicles of a size ranging between 30-120 nm, actively secreted in the extracellular environment by normal as well as tumor cells. Given the increasing understanding of the role of exosomes in cancer progression and the fact that there is an increasing need to improve diagnostics and follow up of the malignancy and growth of tumors, there is accordingly a need for methods and tools to detect and measure exosomes in human fluids. Because of the potential involvement of exosomes in promoting disease progression through a series of detrimental effects on tumor microenvironment, the possibility of quantifying tumor associated exosomes in human plasma or serum, through a sensitive, specific and feasible assay is becoming a crucial issue. If such an assay would be available, it could become a fundamental tool for assessing the potential role of these microvesicles in cancer prognosis, providing a novel prognostic test or a marker for detecting or monitoring neoplastic disease. The currently used methods, e.g. TEM and WB, however are either not, or are only poorly quantitative, whereby there is a clear need for a method to detect and measure exosomes quantitatively in human fluids. Therefore the goal of this disclosure is to provide a new tool for clinical oncologists for diagnosing and follow up studies of cancer patients.

The novel quantitative test that is disclosed here is based on ELISA-mediated detection of TM9SF proteins on exosomes as a reliable test for quantifying and qualifying tumor exosomes (ExoTest™). Notably, TM9SF-proteins have never been shown to be exosome-markers before.

The principle of ExoTest™ is based on capture and quantification of exosomes through the detection of proteins (i.e. Rab proteins) that, although are not exclusively exosome specific, are shared with cytoplasmatic organelles such as endosomes and lysosomes whose membranes are not recycled as for plasma membrane structures. This feature excludes the possibility of detecting these proteins on circulating tumor cells or debris derived from necrotic (tumor) cells or in their soluble form.

The assay of this disclosure includes both tumor markers (such as TM9SF4), which allow preferential detection of tumor-secreted exosomes, and exosomes housekeeping proteins such as CD81, CD9 and CD63. A series of comprehensive studies performed by Western blotting and flow cytometry (FACS) in different experimental conditions as described in the examples below, prove the reliability of the novel test of this disclosure.

TUCAP-1 belongs to the Transmembrane 9 Superfamily (TM9SF), a highly conserved family of proteins characterized by the presence of a large variable extracellular N-terminal domain and nine to ten putative Transmembrane domains. Function and localization of the protein was not described before US Serial Number 2009/0191222 and the corresponding provisional application No. 61/062,453, both of which are fully incorporated herein by reference, which disclosed that TUCAP1-protein is highly expressed in malignant cells, and that the protein was undetectable on cell lines deriving from primary lesions but was present in malignant melanoma cell lines. Moreover, the protein was shown to be involved in the phagocyte behavior of metastatic melanoma cells, since silencing the gene encoding the proteins strongly inhibited the phagocytic behavior of metastatic cells. FIG. 1 shows the molecular structure of the protein. FIG. 3 shows expression of the protein in malignant melanoma cells.

As described in US Serial Number 2009/0220944 and corresponding provisional application 61/062,528, both of which are incorporated herein by reference, ExoTest™ is a fast and efficient ELISA-based test to quantify and characterize exosomes. In this application the concept is broadened to capture and quantify exosomes from human body fluids, in particular circulating plasma exosomes based on expression of housekeeping proteins (CD63 and Rab-5) and TM9SF proteins, utilizing anti-Rab5 or anti-TM9SF antibodies (described in nonprovisional application entitled “Monoclonal antibodies, hybridomas, and methods for use” for Francesco Lozupone, Stefano Fais, Antonio Chiesi, Angela Pontillo, Paolo Sarmientos and Natasa Zarovni, filed on the same day as this application and incorporated herein by reference) for respectively capturing either all exosomes or specifically tumor exosomes present in purified exosome preparations or unfractionated plasma samples. In this application we propose TM9SF4 (SEQ ID NO: 2) as a tumor associated exosomal protein to detect or capture tumor exosomes. We also propose TM9SF1 (SEQ ID NO: 8), TM9SF2 (SEQ ID NO: 4) and TM9SF3 (SEQ ID NO:6) as exosome associated proteins exploitable for capturing or detecting exosomes.

The expression of TM9SF proteins on tumor cells was addressed on several tumor model lines. We have characterized the expression of TM9SF4 protein on malignant melanoma cells, peripheral blood lymphocytes and differentiated macrophage, confirming specific presence of the protein on tumor cells, as shown in FIG. 3. The expression of the protein was also shown on Colo1 (colon carcinoma) cells by FACS and WB (FIGS. 5A and B). In addition, the expression of TM9SF4 (TUCAP1) has been addressed by RT-PCR in different tumor lines comprising B lymphoma, colon carcinoma, breast carcinoma, osteosarcoma, prostate cancer and ovary cancer (FIG. 6).

FIG. 1 shows the molecular structure of TM9SF1, TM9SF2 and TM9SF3-proteins. The expression of the proteins TM9SF1, TM9SF2 and TM9SF3 on tumor cells was characterized on melanoma (MM1) and colon carcinoma (Colo1) cells by FACS, WB and Immunofluorescence analysis (FIGS. 4 A-C). All three proteins were found expressed on the model cell lines.

To address exosome association of TM9SF-proteins, in a first set of experiments we used exosome preparation from conditioned culture media of human tumor cell lines to evaluate the expression of TM9SF-proteins on exosomes by FACS. The results showed that all the proteins belonging to the TM9-Superfamily are detectable on exosomes. As positive controls typical exosomal antigens (CD63 and CD81) were used. (FIG. 7).

Western blot analysis of TM9SF4 (as protein representative of the whole superfamily of these proteins) further confirmed these results, as shown in FIG. 4 where exosomal lysates of MM1 and MM2 metastatic melanoma cell lines were immunoblotted with anti-TM9SF4 antibodies (FIGS. 8A-B) and with the exosomal protein Rab5. (FIG. 4C). As positive controls total cell lysates were immunoblotted with the same antibodies. (FIGS. 8 A-C second and fourth lane). Subsequently, the enrichment of TUCAP1 in exosomal fractions with respect to corresponding whole cell lysates from melanoma (MM1 and MM2), colon cancer (Colo1) and prostate cancer (LnCap) cells was demonstrated in WB using both polyclonal rabbit anti-TUCAP-1 serum (FIG. 9A) and monoclonal anti-TUCAP-1 antibodies (MM1) (FIG. 9B).

Initial analysis of exosomes purified by ultracentrifugation from melanoma patients' plasma confirmed the presence of TUCAP1-protein with its enrichment on tumor patients' plasma exosomes with respect to healthy donor samples (FIG. 10). Moreover, the increased TUCAP 1 expression as well as distinct bands were associated to advanced (stage III/IV) patients according to described role of TUCAP-1 in conferring tumor malignancy. Presence of other TM9SF-proteins on plasma exosomes from another set of melanoma patients with advanced and early tumors was confirmed by FACS analysis (FIG. 11). FACS is hardly a quantitative method and does not enable efficient comparison of protein expression on exosomes from different stage tumor patients and healthy control. This was subsequently addressed by ExoTest™ (see below).

Exosomes purified by ultracentrifugation of supernatants of MM1 and MM2 metastatic melanoma cell lines were also analyzed by ExoTest using anti-TM9SF4 in comparison with the antiCD81 and antiCD63 as exosome detection antibodies. Results shown in FIG. 12 clearly suggest that this protein is detectectable on tumor exosomes analyzed by ExoTest™. Interestingly, on these exosomes TUCAP1 (SEQ ID NO: 2) level of expression, (measured as OD values×1000 wavelength 450 nm), was in average two times higher than values registered for other exosomal markers CD63 and CD81.

Important results were obtained by analyzing by ExoTest™ exosomes purified from plasma of healthy donors as compared to exosomes collected from plasma of melanoma patients. TM9SF4 is strongly detectable on exosomes deriving from melanoma patients' plasma while TMSF4 levels on exosomes of healthy donors do not seem to differ significantly from negative controls. (FIG. 13). Such analysis was then extended to plasma samples obtained from advanced or early disease staged patients (stage III and I respectively) with melanoma, ovary and prostate cancer (FIGS. 15-17). Noteworthy, enrichment of exosomes positive for TM9SF-proteins with respect to overall exosomes is preferentially, though not exclusively, observed in advanced tumor patients and a very high expression is observed in some advanced tumor patients. Some patients presenting a high enrichment of TM9SF1-4 positive exosomes and defined as being on early stage of carcinoma in the clinical info-sheet accompanying the collected sample, may in reality be on more advanced stage than declared due to the scarce precision of the diagnostic method used for patient's staging. The correct staging of the patient may be therefore confirmed only by clinical and laboratory follow-up examinations, for which the instant invention is suitable.

Finally, further test of ExoTest™ method for capture and quantification of overall exosomes from human plasma samples confirmed the suitability of the assay for reliable quantitative analysis from either purified exosomes or unfractionated plasma samples. Noteworthy, when the material deriving from same plasma sample volume was analyzed in parallel, the readings obtained from purified samples corresponded to those from unfractionated samples (FIG. 14). The reliability of the ExoTest assessment of unfractioned samples was confirmed in initial comparative testing of purified plasma exosomes vs. unfractionated plasma samples from a set of tumor patients for the expression of TM9SF1-4 (FIGS. 18 and 19). ExoTest on unfractionated samples maintained fine sensitivity in detection of these markers and produced readings that were in line with what obtained on purified exosomes from the same sample. Noteworthy only 100 μm of unfractionated precleared plasma was used in comparison to 0.5 ml of plasma used for exosome purification.

These results show that while ExoTest™-based on the utilization of CD63 or CD81 as detecting antigen was able to quantify exosomes in plasma of patients with tumors and of healthy subjects, exosomal levels of TM9SF4 is increased in the plasma of tumor patients as compared to plasma of healthy individuals. Accordingly TM9SF4 represents a specific tumor marker, and ExoTest™ using antiTM9SF4-antibodies is a successful test to quantify increase of its expression in particular in malignant tumors. In a similar way other members of TM9SF family, TM9SF1-3, delineate as tumor associated markers. As was suggested in US Serial Number 2009/0220944 and corresponding provisional application 61/062,528, increase in the exosome quantity may correlate with the tumor size. Accordingly, the method and kit provided here can be used to diagnose a tumor and to follow its development.

Altogether, the results show that an exosome-detecting ExoTest™ is working and is useful for detection and quantification of circulating exosomes in humans. Moreover, the test offers a possibility of detecting different proteins in plasma exosome preparations, with a potential application to specific tumor type or a subtype and/or stage. This disclosure also proposes novel potential prognostic/diagnostic tools for tumor patients based on quantification and characterization of plasma exosomes. This is particularly relevant for those tumors that currently lack measurable and reliable prognostic markers. Such is the case, for example with melanoma patients, because the only prognostic serum factor for assessment of disease course and prognosis are LDH (lactate dehydrogenase) levels. The importance of the test according to this disclosure is not limited to melanoma, but can be used for most solid tumors, for which there are currently no measurable and reliable prognostic markers.

We have shown that TM9SF proteins are present in human tumor cells and accordingly in exosomes from tumor patients' fluids and that ExoTest™ using antiTM9SF-antibodies can be used to diagnose and follow up development of tumors in melanoma, prostate and ovary cancer patients. It is evident for one skilled in the art that the same method can be used to detect and follow up any tumors where TM9SF-proteins are expressed.

The invention is now described with non limiting illustrative examples and experimental details are disclosed to provide an improved understanding and guidance for those skilled in the art. The scope of the invention is determined by the appended claims.

### EXAMPLES

### Example 1

### Immunocytochmemistry Shows TM9SF4 Protein Expression in Melanoma Cells

Immunocytochemistry and immunohistochemistry:

For immunocytochemistry melanoma cells and macrophages, cultured on glass chamber slides (Falcon), and PBL, cytospun on glass slides, were fixed with 80% methanol 10 minutes at 4° C. and stained for TUCAP-1, TUCAP-1 mouse serum or preimmune control serum. Malignant melanoma and corresponding normal skin tissue from Biomax array slides (Biomax) were immunostained with pre-immune serum, for anti-TUCAP-1 mouse antiserum. Melanoma was also stained for anti-gp100 (Immunotech) while normal skin was also stained for anti-ezrin (Sigma). Proteins were visualized using the peroxidase antiperoxidase method in single staining (Dako) and counterstained with Mayer's hematoxylin.

FIG. 3A-C shows that MM2 cell lines (A), Peripheral blood lymphocytes (B), and in vitro differentiated Macrophages (C), were negative for mouse preimmune serum. However, malignant melanoma cultured cells showed clear positive staining for TUCAP-1 (FIG. 3D) while PBL (FIG. 3E) and macrophages (FIG. 3F) were negative for TUCAP1 staining. Immunohistochemical analysis of malignant melanoma tissues as compared to healthy skin suggested that TUCAP-1 was detectable only in melanoma tissues (FIG. 3H) while undetectable in healthy skin (3K). As positive control markers for melanoma and normal skin GP100 (FIG. 3I), and ezrin (FIG. 3L) were used respectively. Pre-immune mouse serum staining was always negative in both tissues (FIGS. 3G, 3J). These results provide clear evidence that TUCAP-1 was exclusively detectable in melanoma cells.

### Example 2

### Western Blot, Immunofluorescence and FACSs Studies Show TM9SF4 (TUCAP-1)-Protein Expressing in Colon Carcinoma Cells

Purification of Exosomes Purification from Cell Culture Supernatants and Plasma.

Supernatants from human cell lines were harvested from 72 hours 70-75% confluent cell cultures, and exosomes were isolated as follows. Briefly, after centrifugation of cells at 300 g for 10 minutes, supernatants were centrifuged at 1,200 g for 20 minutes followed by 10,000 g for 30 minutes. Supernatants were filtered using a 0.22 μm filter (Millipore Corp., Bedford, Mass.) and centrifuged at 100,000 g for 1 h in an ultracentrifuge (Sorval) in order to pellet exosomes. Exosomes were washed and resuspended in PBS.

### Western Blotting

Cell lysates were prepared from cells harvested at 70-90% confluency. Cell flasks were washed with PBS, cells detached with Trypsin-EDTA for 2 minutes at room temperature and trypsin quenched with cell growth media containing 5% serum. Cells were washed with PBS and collected by centrifugation at 1500 rpm for 5 minutes at room temperature. Cell lysates are prepared by incubation of cell pellets in TritonX containing lysis buffer and stored at −20° C. till use. Whole cell lysates were resuspended in SDS sample buffer, denatured by boiling, separated by SDS page. After semi dry transfer to nitrocellulose membrane, immunoblotting was performed with indicated primary antibodies for either 0/N at 4° C. or for 2 hours at room temperature. Membrane was thoroughly washed with PBS-Tween and then incubated with suitable HRP conjugated secondary antibody for 1 hour at room temperature. After washing the membrane was incubated with freshly made mix of Cheminoluminescent Substrate A and B for 1 minute and then used to expose and develop the film in the dark room.

Flow Cytometry Analysis of Cell Antigens

Determination of antigen expression on model tumor cell lines was performed by flow cytometry analysis on cells that were harvested at 80-90% confluency. Cell flasks were washed with PBS, cells detached with Trypsin-EDTA for 2 minutes at room temperature and trypsin quenched with cell growth media containing 5% serum. Cells were washed with PBS and collected by centrifugation at 1500 rpm for 5 minutes at room temperature. 10⁴ cells were used per test, resuspended in 100 μm of PBS-FCS. Incubation with primary antibody was done at 4° C. for 45 minutes, cells washed in PBS-FCS by centrifugation 1500 rpm/5 minutes/RT, and incubated with secondary antibody at 4° C. for 30 minutes. After washing in PBS-FCS, cell suspensions are analyzed using BD FACS Calibur. Where needed cells were permeabilized before incubation with primary antibody by fixation in 4% PFA (paraformaldehyde) and subsequent treatment with 0.01% TrytonX for 10 minutes at 4° C.

As an addition to previous analysis of melanoma cells for the expression of TM9SF4 (TUCAP-1) FIG. 5. shows its expression on another human tumor cell line model. The protein is detected on Colo1 cells with FACS (FIG. 5A), WB (FIG. 5B) and Immunofluorescence (FIG. 5C) using both polyclonal rabbit serum anti TM9SF4 and some monoclonal anti TM9SF4-antibodies of in-house obtained panel.

### Example 3

### RT-PCR Analysis Shows TUCAP-1 Protein to be Expressed in Prostate Cancer, Osterocarcoma, B Cell Lymphoma, Breast Carcinoma, and Ovary Carcinoma Cell Lines

PCR Analysis.

Expression of Tucap-1 transcripts was assessed by RT-PCR on several tumor cell lines. Total RNA from the cells was obtained by the RNAzol (Invitrogen) method and RNA templates were used for RT-PCR amplification.

| Primers for TUCAP-I detection were: | |
|---|---|
| tgtgtgaaacaagcgccttc, | (SEQ ID NO: 9) |
| and | |
| atgaggtggacgtagtagt. | (SEQ ID NO: 10) |
| These primers amplify a fragment of | |
| 349 base pairs. | |
| Primers to detect GAPDH were: | |
| ccatggagaaggctgggg | (SEQ ID NO: 11) |
| and | |
| caaagttgtcatggatgacc. | (SEQ ID NO: 12) |

The suggested feature of TUCAP-1 as a tumor marker is strongly supported by a reported expression of a corresponding mRNA in a wider panel of human malignant cancer cell lines including B lymphoma, breast carcinoma, prostate cancer and ovary carcinoma, as is demonstrated on FIG. 6.

### Example 4

### Analysis of TM9SF Proteins on Tumor Cell Lines

Two tumor cell lines, MM1 and Colo1 (melanoma and colon cancer respectively) were used to assess the expression of TM9SF1 (SEQ ID no:8), TM9SF2 (SEQ ID NO:4) and TM9SF3 (SEQ ID NO:6). In-house produced panel of monoclonal antibodies is used in these experiments. FIG. 4. shows expression of all three proteins on tumor cells, as obtained with FACS (FIG. 4 A), WB (FIG. 4.B) and Immunofluorescence (FIG. 4.C) suggesting tumor association to all family members. FIG. 5 shows similar results of TMSF4-protein. The results disclosed here thus clearly proves that all the proteins of TM9-superfamily are expressed on tumor cells.

### Example 5

### FACS Analysis of TM9SF Proteins on Tumor Exosomes

Cell Cultures

Two types of human tumor cell lines were used, i.e. melanoma and colon carcinoma. MM1 and MM2 are two metastatic melanoma cell lines obtained from metastatic lesions of patients, surgically resected. Colo is a colorectal carcinoma cell line derived from a liver metastasis of colorectal cancer patient. All cell lines were cultured in RPMI 1640 medium supplemented with 100 IU/ml penicillin, 100 μg/ml streptomycin (Gibco), 2 mM glutamine (Gibco) and 10% fetal calf serum (FCS) (Invitrogen, Milan, Italy).

Flow Cytometry Analysis of Exosomes

Determination of antigen expression on exosomes was performed by flow cytometry analysis on purified exosomes bound onto latex beads. Exosome preparations (5-10 μg) were incubated with 5 μl 4-μm-diameter aldehyde/sulfate latex beads (Interfacial Dynamics, Portland, Oreg.) and resuspended into 400 μm PBS containing 2% FCS. Exosomes-coated beads (20 μl) were incubated with the in house produced anti-TM9SF proteins antibodies and with antibodies against known exosomal markers: anti-CD63-FITC (Pharmigen) and anti-CD81-PE (Pharmingen) for 30 minutes at 4° C., followed, when needed, by incubation with PE- or FITC-conjugated secondary antibody and analyzed on a FACSCalibur flow cytometer (BD Biosciences).

Culture supernatants of melanoma cell lines were processed following the standard procedure to obtain purified exosomes as described above. Exosomes bound to latex beads were analyzed by FACS. In order to verify if TM9SF proteins (TM9SF1, TM9SF2, TM9SF3 and TM9SF4) can be detectable on tumor deriving exosomes, we started to analyze by FACS TM9SF proteins expression on exosomes collected from supernatants of in vitro cultured Colo cells and MM1 cells (not shown). Results represented in FIG. 4 clearly suggest that all proteins belonging to this family are detectable on tumor derived exosomes.

As positive control exosomes were stained for CD63 and CD81 two widely used exosomal markers. AlexaFluor488 conjugated goat anti-mouse secondary antibody staining was utilized as negative control.

### Example 6

### Western Blot Analysis of TM9SF4 on Tumor Cells and Corresponding Exosomes

Western Blot Analysis of Exosomes

Purified exosomes were lysed in lysis buffer containing 1% Triton X-100, 0.1% SDS, 0.1 M Tris HCl (pH 7) and protease inhibitors (10 μg/ml aprotinin, 10 μg/ml leupeptin and 2 mM phenylmethylsulfonyl fluoride) (Sigma). Exosome protein concentration was determined by Bradford microassay method (Bio-Rad Laboratories, Hercules, Calif.). A total of 50 μg of proteins was resuspended in SDS sample buffer, boiled for 5 min, separated on 10% SDS-PAGE gel and electroblotted on nitrocellulose (Protran BA85, Schleicher and Schuell). Membranes were blotted with antibodies to TM9SF4 (diluted 1:50) and Rab-5b (diluted 1:50), incubated with appropriate HRP-conjugated secondary antibodies (Amersham Pharmacia) and visualized by enhanced chemiluminescence (ECL, Pierce).

Western blot analysis of TM9SF4 on exosomes (FIG. 8. lanes 1 and 3) purified from supernatants of in vitro cultured MM1 and MM2 cells and on MM1 and MM2 total lysates (FIG. 8, lanes 2 and 4), show that in these samples TM9SF4 is detectable on exosomes and on whole lysates of both cell lines, further suggesting that this protein can be considered a tumor marker. Rab5 was used as an exosomal marker. These results confirm FACS analysis of exosomes deriving from the same cell lines. In addition to MM1 and MM2 cells, TM9SF4 was detected on both whole cell lysates and exosomes derived from Cobol and LnCap cell supernatants (FIG. 9.A). Noteworthy, using a panel of in house made monoclonal anti-TM9SF4 antibodies the enrichment of TM9SF4 in exosomal fraction is observed when comparing the same amounts of MM1 whole cell lysate and exosome lysate (FIG. 9.B).

### Example 7

### Western Blot Analysis of TM9SF4 on Melanoma Patients' Plasma Exosomes

Human Donors and Tumor Patients' Plasma: Human plasma samples were collected from EDTA-treated whole blood from patients with primary or metastatic melanoma and from age and sex-matched healthy donors. Samples were stored at −70° C. until analysis.

In order to obtain exosomes from plasma samples, heparinized blood from tumor patients and healthy donors were centrifuged at 400×g for 20 minutes. Plasma was then collected, aliquoted and stored at −70° C. until analysis. Plasma samples were subjected to the same centrifugal procedure described above to isolate exosomes by using a Beckman TL100 for ultracentrifugation of small volumes.

Initial assessment of association of TM9SF4 to tumor derived circulating exosomes was performed by WB analysis of human plasma exosomes purified from plasma samples from patients with melanoma and from healthy donors pool. Exosomes purified from 0, 5 ml of plasma were loaded per lane MM1 exosomes (mexo) that were already known to carry TUCAP-1, were used as a control. Evident increase of TUCAP-1 on tumor patients' exosomes was observed when compared to Healthy donor (HD) sample. Increased expression of exosome associated TUCAP along with a specific presence of distinct bands, is observed in advanced (stage III/IV) patients differently from early cancer patients (stage I/II).

These results demonstrate the TUCAP-1 association to plasma exosomes from cancer patients. Level and a pattern of expression are dependent on the stage of disease.

### Example 8

### FACS Analysis of TM9SF Proteins on Plasma Exosomes from Melanoma Patients

Initial assessment of association of TM9SF1-3 proteins to tumor derived circulating exosomes was performed by FACS analysis of human plasma exosomes purified from plasma samples from patients with melanoma and from healthy donors pool. For these analyses monoclonal anti TM9SF1-3 antibodies produced in-house were used that already recognized the proteins of interest on Colo1 exosomes. Exosomes purified from plasma were conjugated to latex beads and analyzed as described above. Single test was performed with exosomes corresponding to 0.25 ml of plasma sample. TM9SF proteins were present in all patients sample as well as to a lesser extent, on HD pool. FACS is poorly quantitative and though confirming the presence of TM9SF proteins on tumor exosomes does not enable comparative quantification and does not allow us to appreciate eventual differences between different stage patients or between patients and healthy controls.

### Example 9

### Antibodies Against TM9SF-Proteins

In order to produce polyclonal antibodies to TM9SF4 (TUCAP-1), cDNA from MM1 cells were cloned in bacterial expression vectors to obtain TUCAP-1 amino acids 18-279 (SEQ ID NO: 13) fused to a 10-Histidine N-terminal tag (SEQ ID NO: 14). Purified recombinant peptide was used to produce anti-TUCAP-1 antibodies in mice. The anti-TUCAP-1 antibodies recognized immunogen, GFP-tagged full length protein as positive control as well as endogenous TUCAP-1 protein.

Polyclonal antibodies were also generated by immunizing a rabbit with a purified peptide fragment having an amino acid sequence according to SEQ ID NO: 15. The antibodies generated were able to recognize human TUCAP-1 protein by binding to a peptide fragment that consists of amino acids 221-235 of SEQ ID NO: 2. Polyclonal antibodies are also obtained by immunizing a goat and a donkey.

Polyclonal antibodies were further generated by immunizing rabbit with a purified peptide fragment having an amino acid sequence according to SEQ ID NO:16. The antibodies generated were able to recognize human TUCAP 1 protein by binding to a peptide fragment that consists of amino acids 303-352 of SEQ ID NO:2.

Polyclonal antibodies against TM9SF1 were produced similarly using amino acids 90-215 of SEQ ID NO:8 (SEQ ID NO: 17) fused to a 10-Histidine N-terminal tag (SEQ ID NO:14).

Polyclonal antibodies against TM9SF2 were produced similarly using amino acids 106-271 of SEQ ID NO:4 (SEQ ID NO:18) fused to a 10-Histidine N-terminal tag (SEQ ID NO:14).

Polyclonal antibodies against TM9SF3 were produced similarly using amino acids 29-222 of SEQ ID NO: 6 (SEQ ID NO:19) fused to a 10-Histidine N-terminal tag (SEQ ID NO:27).

Production of monoclonal antibodies is described in details in the co-pending application entitled “Monoclonal antibodies, hybridomas, and methods for use” for Francesco Lozupone, Stefano Fais, Antonio Chiesi, Angela Pontillo, Paolo Sarmientos, and Natasa Zarobvni, filed on the same day as this application and fully incorporated by reference. Selected hybridoma clones were generated by using spleen cells of selected mice. Briefly B-cells deriving from spleen of immunized mice were fused with a myeloma tumor cell lien specifically selected from hybridoma production. The reviving fused (hybrid) cells that can grow indefinitely in culture with consequent production of large amounts of the desired antibodies. Hybridoma production is performed according to standard protocols. After screening the selected hybridomas, the hybridomas are cloned and grown to large-scale for antibody productions. Various hybridomas are selected for various purposes including laboratory use, preclinical and clinical studies and tumor diagnosis and prognosis tools, such as detection kits. The monoclonal antibodies produced bind to conformational or linear epitopes of TUCAP 1 protein amino acids 18-279 of SEQ ID NO:2, or peptide frame consisting of amino acids 221-235 or consisting of amino acids 303-352 of SEQ ID NO:2.

### Example 10

### ExoTest™ Analysis of Purified Exosomes from Cultured Cells Supernatants

Culture supernatants of melanoma and colon carcinoma cell lines were processed following the standard procedure to obtain purified exosomes as described above. ExoTest™ performed on exosomes purified by ultracentrifugation of supernatants of MM1 and MM2 metastatic melanoma cell lines analyzed for the detection of TM9SF4 clearly show that this protein is highly expressed on exosomes of tumor cells, and TM9SF4 level of expression on exosomes is higher than CD81 and CD63, two acknowledged exosome related proteins (FIG. 12).

Negative control: Rab5 coated wells plus detecting antibodies (antibodies to TM9SF4 or CD81 or CD63) and secondary antibody. Exosomal proteins levels are expressed as OD (wavelength 450 nm)×1000.

### Example 11

### ExoTest™ Analysis for TM9SF4 Expression on Exosomes Derived from Plasma Samples

ExoTest™ analysis of TM9SF4:

Basic ExoTest™ has been described in US nonprovisional application Ser. No. 12/231,412 and in corresponding provisional application 61/062,528, both of which are incorporated herein by reference. Briefly, exosomes purified as described before, were added into anti Rab-5 rabbit pAbs coated ninety-six well-plates (HBM) and incubated overnight at 37° C.

After washings with PBS, mouse anti-TM9SF4 antibody 1A4 or mouse anti CD63 and CD81 (Pharmingen) antibodies were added, as detection antibodies. In subsequent assays mouse anti-TM9SF1, -TM9SF2 and -TM9SF3 were used (clones 10A11, 2D2 and 2C7-E2 respectively). After washings PBS, the plate was incubated with HRP-conjugated anti-mouse-peroxidase secondary antibody (Pierce) and the reaction was developed with POD (Roche), blocked with 1N H2504. As negative control, Rab5 coated wells incubated with detecting antibodies followed by secondary antibodies, was used. Optical densities were recorded with an ELISA reader by using a 450 nm filter (Biorad).

Exosomes were purified from plasma of three different melanoma patients (affected by advance disease stage III-IV) and three healthy donors and were then subjected to ExoTest™ for TM9SF4 and CD63 detection. Negative control: Rab5 coated wells plus detecting antibodies (antibodies to TM9SF4 or CD63) and secondary antibody. Exosomal proteins levels are expressed as OD (wavelength 450 nm)×1000. Quantification of exosomes based on TM9SF4 expression by ExoTest is shown in FIG. 6 that clearly shows that: i) TM9SF4 antibodies have a higher sensitivity for the detection of tumor exosomes when compared with CD63; ii) TM9SF4 values of obtained exosome samples of healthy donors plasma are comparable to negative controls. Accordingly we suggest here that circulating TM9SF4 may be associated to exosomes in melanoma patients, and quantification of plasma exosomes bearing this protein may be considered a useful tumor marker.

TM9SF4 positive exosomes were next quantified in both patients with early disease or advanced melanoma, ovary or prostate tumors. Results in FIGS. 15-17 are shown as RLU values as luminometric detection was used for assay development. Initial testing on a limited patient group showed elevated level of TM9SF4 expression in patient samples analyzed with respect to healthy donors pool derived exosomes. Exosome associated TM9SF4 could be a useful marker for accurate tumor staging.

### Example 12

### ExoTest™ Analysis for TM9SF1-3 Expression on Exosomes Derived from Plasma Samples

Beside TM9SF4 protein also TM9SF1-3 were quantified on exosomes purified from a set o plasma samples from patients with ovary, prostate cancer and melanoma FIGS. 15-17. CD63 quantification was used for overall exosome quantification in the sample and estimate of enrichment of TM9SF positive exosomes in the sample. Among analyzed patient samples, some had significantly increased levels of TM9SF proteins, mostly corresponding to advanced tumor stage, but also to some patient samples staged as early disease. No patients follow up information was available at the time the test was performed. The sensitivity and reproducibility of the test was high. This indicates a potential relevance of TM9SF proteins on tumor exosomes for accurate tumor staging and monitoring.

### Example 13

### Inverted ExoTest for Analysis for Expression of TM9SF-Proteins on Exosomes Derived from Plasma Samples

The basic ExoTest that was originally disclosed and described in US Serial Number 2009/0220944 is a versatile assay that allows different combinations of capturing and detection of antibodies. In the original kit we first capture the exosomes with an antibody against a housekeeping protein, such as Rab5 and the detection antibody is an anti-TMSF9-antibody. We have also developed an ‘inverted’ ExoTest, where the exosomes derived from plasma samples are first captured by using anti-TM9SF-antibodies, and the detection antibody is an antibody against a housekeeping protein, such as Rab5. Alternatively the detection antibody may be anti-Cav1-antibody or anti-CD63-antibody.

### Example 12

### Quantification of Exosomes by Using Unfractionated Biological Fluids

In order to provide a test for clinical purposes it was necessary to verify that the test could be used for exosome detection in unfractionated biological fluids that would allow an easy and reproducible analysis avoiding the steps of ultracentrifugation. We compared the detection and quantification of CD63+exosomes from unfractionated samples (cell culture supernatants from human macrophages and melanoma cells, and human plasma) and exosomes purified from the same samples. In order to increase the sensitivity of the test, for the specific experiments the HRP-conjugated Mab was incubated for 30 minutes instead of 15 minutes. The presence of exosomes from unfractionated macrophages and melanoma culture supernatants and plasma from nine melanoma patients was detectable by ExoTest (results not shown). In addition we performed the same analysis of plasma from 4 healthy donors and regression analysis on the total number of samples analyzed (9 patients+4 healthy donors) showed a significant correlation between the two types of measures (results not shown). These results suggest that ExoTest is useful and reliable in clinical setting using whole plasma and avoiding the complex and time consuming procedures of exosome purification. This notion is further reinforced by comparative ExoTest-quantification of CD63 in unfractionated plasma samples pre-cleared by microfiltration through 0.22 μm filters and concentrated by using 100K cut off spin concentrators (Millipore) analyzed side by side with exosomes purified from the same volume of the same sample. As demonstrated in FIG. 14, highly comparable OD readings for a single sample were obtained. Finally, comparative quantification of TM9SF1-4 by ExoTest on purified plasma exosomes vs. unfractioned plasma samples from patients with melanoma, ovary and prostate cancer revealed high sensitivity and reproducibility of the assay on unfractioned plasma samples. Same set of patients was analyzed for the CD63 expression for the purpose of overall exosomes quantification and for the presence and enrichment of TM9SF4 (FIG. 18) and TM9SF1-3 (FIG. 19) positive exosomes. Standard ExoTest and sample purification/preclearing protocols were used and the ExoTest developed by colorimetric or luminometric detection and results shown as OD 450 nm or RLU readings.

### Example 13

### Method to Diagnosis and Prognosis

Although exosomes are released by diverse if not all proliferating cell types, their release is exacerbated in tumor cells, as evidenced by their increased presence in plasma, ascites, and pleural effusions of patients with cancer [8, 7, 14]. Moreover the correlation of circulating exosomes and a size of a tumor was demonstrated using ExoTest for plasma exosomes quantification in U.S. Serial Number 2009/0220944 and corresponding provisional application No. 61/062,528, and in the subsequent publication. (Logozzi et al 2009) all of which are fully incorporated herein by reference.

Based on the results shown in this disclosure, it would be evident for one skilled in the art that TM9SF-proteins are useful tumor markers and that present on tumor exosomes. The examples presented here are related to melanoma tumors, colon cancer tumors, prostate cancer tumors, osteosarcoma tumors, B cell lymphoma tumors, breast cancer tumors and ovary carcinoma tumors. However, one skilled in the art would understand that the method described here would be useful in detecting any other cancer types where TM9SF-proteins are expressed. Such other cancer types could include lung cancer, bladder cancer, gastrointestinal cancers, and brain tumors.

Sequences table:

| Sequence | |
|---|---|
| number | Description |
| SEQ ID NO 1 | TM9SF4: Encoding sequence for the full protein |
| SEQ ID NO 2 | TM9SF4: Amino acid sequence for the full protein |
| SEQ ID NO 3 | TM9SF2: Encoding sequence for the full protein |
| SEQ ID NO 4 | TM9SF2: Amino acid sequence for the full protein |
| SEQ ID NO 5 | TM9SF3: Encoding sequence for the full protein |
| SEQ ID NO 6 | TM9SF3: Amino acid sequence for the full protein |
| SEQ ID NO 7 | TM9SF1: Encoding sequence for the full protein |
| SEQ ID NO 8 | TM9SF1: Amino acid sequence for the full protein |
| SEQ ID NO 9 | TM9SF4: Primer for TM9SF4 detection - forward |
| SEQ ID NO 10 | TM9SF4: Primer for TM9SF4 detection - reverse |
| SEQ ID NO 11 | GAPDH: Primer for GAPDH detection - forward |
| SEQ ID NO 12 | GAPDH: Primer for GAPDH detection - reverse |
| SEQ ID NO 13 | TM9SF4: Amino acid sequence for His |
| | tagged TM9SF4 aa 18-279 |
| SEQ ID NO 14 | p2N N-terminal His Tag amino acid sequence |
| SEQ ID NO 15 | TM9SF4: Amino acid sequence corresponding |
| | to TM9SF4 aa 221-235 |
| SEQ ID NO 16 | TM9SF4: Amino acid sequence corresponding |
| | to TM9SF4 aa 303-352 |
| SEQ ID NO 17 | TM9SF1: Amino acid sequence corresponding |
| | to TM9SF1 aa 90-215 |
| SEQ ID NO 18 | TM9SF2: Amino acid sequence corresponding |
| | to TM9SF2 aa 106-271 |
| SEQ ID NO 19 | TM9SF3: Amino acidic sequence corresponding |
| | to TM9SF3 aa 29-222 |

### REFERENCES

1. Thery C, Zitvogel L, Amigorena S. Exosomes: composition, biogenesis and function. Nat Rev Immunol. 2002 August; 2:569-79.
2. Stoorvogel W, Kleijmeer M J, Geuze H J, Raposo G. The biogenesis and functions of exosomes. Traffic. 2002 May; 3(5):321-30.
3. Raposo G, Tenza D, Mecheri S, Peronet R, Bonnerot C, Desaymard C. Accumulation of major histocompatibility complex class II molecules in mast cell secretory granules and their release upon degranulation. Mol Biol Cell 1997; 8:2631-45.
4. Heijnen H F G, Schiel A E, Fijnheer R, Geuze H J, Sixma J J. Activation platelets release two types of membrane vesicles: microvesicles by surface shedding and exosomes derived from exocytosis of multivesicular bodies and alpha granules. Blood 1999; 94:3791-9.
5. Taylor D D, Black P H. Shedding of plasma membrane fragments: Neoplastic and developmental importance. In: Steinberg M, editor. Developmental Biology, vol. 3; 1986. p. 33-57.
6. Taylor D D, Bohler H C, Gercel-Taylor C. Pregnancy-linked suppression of TcR signaling pathways by a circulating factor absent in recurrent spontaneous pregnancy loss. Molecular Immunology 2006; 43:1872-80.
7. Andre F, Schartz N E, Movassagh M, et al. Malignant effusions and immunogenic tumour-derived exosomes. Lancet 2002; 360:295-305.
8. Valenti R, Huber V, Filipazzi P, Pilla L, Sovena G, Villa A, et al. Human tumor-released microvesicles promote the differentiation of myeloid cells with transforming growth factor-beta-mediated suppressive activity on T lymphocytes. Cancer Res 2006; 66:9290-8.
9. Olver C, Vidal M. Proteomic analysis of secreted exosomes. Subcell Biochem 2007; 43:99-131.
10. Mears R, Craven R A, Hanrahan S, et al. Proteomic analysis of melanomaderived exosomes by two-dimensional polyacrylamide gel electrophoresis and mass spectrometry. Proteomics 2004; 4:4019-31
11. Bard M P, Hegmans J P, Hemmes A, et al. Proteomic analysis of exosomes isolated from human malignant pleural effusions. Am J Respir Cell Mol Biol 2004; 31:114-21.
12. Choi D S, Lee J M, Park G W, et al. Proteomic analysis of microvesicles derived from human colorectal cancer cells. J Proteome Res 2007; 6: 4646-55.
13. Escola J M, Kleijmeer M J, Stoorvogel W, Griffith J M, Yoshie O, Geuze H J. Selective enrichment of tetraspan proteins on the internal vesicles of multivesicularendosomes and on exosomes secreted by human B-lymphocytes. J Biol Chem 1998; 273:20121-7.
14. Logozzi M, De Milito A, Lugini L, Borghi M, Calabra L, Spada M, Perdicchio M, Marino M L,
Federici C, lessi E, Brambilla D, Venturi G, Lozupone F, Santinami M, Huber V, Maio M, Rivoltini L, Fais S. High levels of exosomes expressing C D63 and caveolin-1 in plasma of melanoma patients. PLoS One. 2009; 4(4):e5219.
15. Mathivanan S, Lim J W, Tauro B J, Ji H, Moritz R L, Simpson R J. Proteomicsanalysis of A33 immunoaffinity-purified exosomes released from the human colon tumor cell line LIM1215 reveals a tissue-specific protein signature. Mol Cell Proteomics. 2010 (2):197-208.
16. Mears R, Craven R A, Hanrahan S, Totty N, Upton C, Young S L, Patel P, Selby P J, Banks R E. Proteomic analysis of melanoma-derived exosomes by two-dimensional polyacrylamide gel electrophoresis and mass spectrometry. Proteomics. 2004
17. Zaravinos A, Lambrou G I, Boulalas I, Delakas D, Spandidos D A. Identification of common differentially expressed genes in urinary bladder cancer. PLoS One. 2011 Apr. 4; 6(4):e18135.
18. He P, Peng Z, Luo Y, Wang L, Yu P, Deng W, An Y, Shi T, Ma D. High-throughput functional screening for autophagy-related genes and identification of T M9S F1 as an autophagosome-inducing gene. Autophagy. 2009 5: :52-60.
19. Chang H, Jeung H C, Jung J J, Kim T S, Rha S Y, Chung H C. Identification of genes associated with chemosensitivity to SAHA/taxane combination treatment in taxane-resistant breast cancer cells. Breast Cancer Res Treat. 2011 125: 55-63.
20. Mackinnon R N, Selan C, Wall M, Baker E, Nandurkar H, Campbell L J. The paradox of 20q11.21 amplification in a subset of cases of myeloid malignancy with chromosome 20 deletion. Genes Chromosomes Cancer. 2010 49: 998-1013
21. Lozupone F, Perdicchio M, Brambilla D, Borghi M, Meschini S, Barca S, Marino M L, Logozzi M, Federici C, lessi E, de Milito A, Fais S. The human homologue of Dictyostelium discoideum phg1A is expressed by human metastatic melanoma cells. EMBO Rep. 2009 10: 1348-54

What is claimed is:

1. A method to quantify and qualify tumor-related exosomes in human cell derived samples or in body fluid, said method having the steps comprising:
a) optionally purifying an exosome preparation from the human cell derived sample or body fluid;
b) capturing exosomes of the purified exosome preparation or the human cell derived sample or body fluid with a primary capturing antibody against a housekeeping protein present on exosomes, said primary capturing antibody being selected from the group consisting of: anti-tetraspanins, anti-annexins and anti-Rab-proteins;
c) detecting tumor-related exosomes from the captured total exosomes with a detection antibody, said detection antibody being selected from the group consisting of antibodies against proteins belonging to Transmembrane-9 superfamily;
d) allowing an enzyme linked secondary antibody react with the detection antibody;
e) adding substrate; and
f) detecting the reaction.

2. The method of claim 1, wherein the detection antibody is selected from the group consisting of anti-TM9SF1, anti-TM9SF2, anti-TM9SF3 and anti-TM9SF4-antibody.

3. The method of claim 1, wherein the primary capturing antibody is selected from the group consisting of anti-Rab5-antibody, anti-CD63-antibody, anti-CD9-antibody, and anti-Rab7-antibody.

4. The method of claim 1, wherein the primary capturing antibody is anti-Rab5 antibody and the detection antibody is anti-TMSF4-antibody.

5. The method of claim 4, wherein the anti-TMSF4-antibody recognizes a peptide sequence consisting of amino acids 18-279 of SEQ ID NO:2, amino acids 221-235 of SEQ ID NO:2 or amino acids 303-352 of SEQ ID NO:2.

6. A method to quantify and qualify tumor-related exosomes in human cell derived samples or in body fluid, said method having the steps comprising:
a) optionally purifying an exosome preparation from the human cell derived sample or body fluid;
b) capturing exosomes of the purified exosome preparation or the human cell derived sample or body fluid with a primary capturing antibody, said primary capturing antibody being selected from the group consisting of: antiTM9SF1-, antiTM9SF2-, antiTM9SF3- and antiTM9SF4-antibodies;
c) detecting tumor-related exosomes from the captured exosomes with a detection antibody, said detection antibody being an antibody against a housekeeping protein:
d) allowing an enzyme linked secondary antibody react with the detection antibody;
e) adding substrate; and
f) detecting the reaction.

7. The method of claim 6, wherein the detection antibody is selected from the group consisting of anti-Rab5-, anti-CD63-, and anti-Cav-1-antibodies.

8. A method to diagnose malignant tumor, said method comprising the steps of:
a) taking a body fluid sample of a human subject suspected to have a tumor;
b) optionally purifying an exosome preparation from the sample;
c) capturing exosomes of the purified exosome preparation or the body fluid sample according to step b) of claim 1;
d) detecting the captured exosomes according to step c) of claim 1;
e) allowing an enzyme linked secondary antibody react with the detection antibody;
f) adding substrate;
g) detecting the reaction; and
h) making a correlation between a positive reaction and an expression level of the protein belonging to transmembrane-9 Superfamily and presence of malignant tumor.

9. The method of claim 8, wherein the protein belonging to transmembrane-9 Superfamily is TM9SF1, TM9SF2, TM9SF3 or TM9SF4.

10. The method of claim 8, wherein the primary capturing antibody is anti-Rab 5b-antibody and the detection antibody is selected from the group consisting of anti-TM9SF1, TM9SF2, TM9SF3 and TM9SF4-antibodies.

11. The method of claim 8, wherein the tumor is a human tumor expressing one or more TM9SF proteins.

12. The method of claim 10, wherein the tumor is melanoma tumor, colon cancer tumor, prostate cancer tumor, osteosarcoma tumor, B cell lymphoma tumor, breast cancer tumor or ovary carcinoma tumor.

13. A non-invasive method to monitor tumor growth, said method comprising the steps of:
a) periodically taking a body fluid sample of a patient;
b) optionally purifying an exosome preparation from the samples;
c) capturing exosomes of the purified exosome preparations or the body fluid samples according to steps b) of claim 1;
d) detecting the captured exosomes according to step c) of claim 1;
e) allowing an enzyme linked secondary antibody react with the detection antibody;
f) adding substrate;
g) detecting the reaction; and
h) drawing a correlation between quantity of detected exosomes and size and/or invasiveness of the tumor.

14. The method of claim 13, wherein the tumor is melanoma tumor, colon cancer tumor, prostate cancer tumor, osteosarcoma tumor, B cell lymphoma tumor, breast cancer tumor or ovary carcinoma tumor.

15. A non-invasive method to monitor tumor growth, said method comprising the steps of:
a) periodically taking a body fluid sample of a patient;
b) optionally purifying an exosome preparation from the body fluid sample;
c) capturing exosomes of the purified exosome preparation or the body fluid sample with a primary capturing antibody, said primary capturing antibody being selected from the group consisting of: antiTM9SF1-, antiTM9SF2-, antiTM9SF3- and antiTM9SF4-antibodies;
d) detecting tumor-related exosomes from the captured exosomes with a detection antibody, said detection antibody being selected from the group consisting of anti-Rab5-, anti-CD63 and anti-Cav-1-antibodies;
e) allowing an enzyme linked secondary antibody react with the detection antibody;
f) adding substrate;
g) detecting the reaction, and
h) drawing a correlation between quantity of detected exosomes and size and/or invasiveness of the tumor.

16. A test kit for quantifying and qualifying exosomes in human cell derived samples or in body fluid, said kit comprising:
a) instructions to optionally purify an exosome preparation from the human cell derived sample or from body fluid;
b) a primary antibody preparation for capturing exosomes of a purified exosome preparation or a human body fluid sample;
c) a detection antibody preparation for detecting bound exosomes;
d) an enzyme linked secondary antibody preparation for reaction with the detection antibody;
e) a substrate for the enzyme;
f) a positive control consisting of a standard exosome preparation from a human cancer cell line expressing TM9SF protein(s) of interest; and
g) instructions to compare the reaction of the sample with the reaction of the positive control.

17. The test kit of claim 16, wherein the primary capturing antibody is anti-Rab 5b antibody and the detection antibodies are antiTM9SF1-, antiTM9SF2-, antiTM9SF3- or antiTM9SF4-antibodies.

18. The test kit of claim 16, wherein the primary capturing antibody is selected from a group consisting of anti-TM9SF1-, anti-TM9SF2-, anti-TM9SF3- and anti-TM9SF4-antibody and the detection antibodies are anti-Rab5-, anti-CD63-, or anti-Cav-1-antibodies.

19. A method to capture exosomes from a purified exosomes preparation, a human cell derived sample or a body fluid by allowing the preparation of the sample to react with antibodies against a transmembrane 9-superfamily protein.

20. The method of claim 19, wherein the antibody is selected from the group consisting of anti-TM9SF1; anti-TM9SF2; anti-TM9SF3, and anti-TM9SF4.
